(19) United States
(12) Patent Application Publication
(10) Pub. No.: US 2010/0311107 A1
(43) Pub. Date: Dec. 9, 2010

(54) Ultra-High Throughput Screening of Natural Products

(76) Inventors: Richard H. Baltz, Lincoln, MA (US); Catherine Monahan, West Roxbury, MA (US); Christopher Murphy, Upton, MA (US); Julia Penn, Maidenhead (GB); Daniel Ritz, Luzern (CH); Stephen Wrigley, High Wycombe (GB)

Correspondence Address: K&L Gates LLP, STATE STREET FINANCIAL CENTER, One Lincoln Street, BOSTON, MA, 02111-2950, US

(21) Appl. No.: 12/844,046

(22) Filed: Jul. 27, 2010

Related U.S. Application Data
(62) Division of application No. 11/793,168, filed on Jun. 14, 2007.
(60) Provisional application No. 60/637,141, filed on Dec. 17, 2004.

(51) Int. Cl. C12Q 1/18

(52) U.S. Cl. 435/32

(57) ABSTRACT

The present invention provides cells having more than two drug resistance genes and at least two different resistance genes that have been recombined into the chromosome of a cell. It also teaches the processes for preparing cells by recombining two or more different drug resistance genes into the chromosome of a cell. The invention further shows a screening method using the cells of described herein that may be used to accomplish high throughput screening of, among other things, natural products and/or whole cells isolated from the environment.

### 1. FIELD OF THE INVENTION

The inventions introduced in this application relate to methods of ultra-high throughput screening of natural bioactive products from environmental samples, and to microorganism strains and compositions related to these screening methods.

### 2. BACKGROUND OF THE INVENTION

2.1 Microorganisms: a Valuable Source of Bioactive Natural Compounds

Throughout the past 60 years, the use of natural antimicrobial agents to treat infectious diseases has been one of the most significant successes in medicine. As a matter of fact, 39% of the total 520 new drugs approved from 1983 to 1994 were either natural products or derivatives of natural compounds and 60 to 80% of new antibacterial and anticancer drugs were derived from natural products. Harvey A., 2000, “Strategies for discovering drugs from previously unexplored natural products,” Drug Discovery Today, 5:294-300. It has been estimated that the use of antibiotics has saved more lives than any another medical therapy. Examples of discovered antibiotics, their natural sources and therapeutic applications are summarized in Table 1.

Actinomycetes, which only comprise a small proportion of the whole microbial population, are an essential productive source for bioactive natural compounds, such as antibiotics. Actinomycetes produce compounds with a variety of properties, for example, antibacterial agents (β-lactams, glycopeptides, aminoglycosides, macrolides, lipopeptides), antifungal agents (amphotericin B, candicidin, pimaricin, nystatin), antiviral agents (complestatin, concanamycin, pentostatin), antitumor agents (adriamycin, bleomycin, daunomycin, mithramycin, tetracenomycin), immune modulation agents (FK506, rapamycin, ascomycin), insecticides (spinosad, nanchangmycin), anthelmintic (avermectins, milbemycin, meilingmycin) and anticoccidial agents (monensin, naracin, salinomycin). See Table 1, actinomycete species and the natural compounds they produce are typed in bold. For the reasons mentioned above, actinomycetes are, in most cases, the preferred source for screening for useful natural compounds in the pharmaceutical industry.

Although many antibiotics have already been discovered from environmental sources, especially from microorganisms, such as actinomycetes, and have been used for the treatment of a variety of diseases, the medical community faces a serious concern that bacterial resistance to commonly used antibacterial antibiotics is emerging rapidly. Similarly, a rapid increase of resistance to antifungal agents has also been observed. Due to the modest efficacy of available antifungals against life-threatening systemic fungal infections and the rapidly increasing resistance to these antifungal agents, it is of specific interest to discover new classes of antifungal agents. The pharmaceutical industry has responded to this threat, in part, by seeking to develop or discover structurally novel antibiotics to thwart increased antibiotic resistance. For many decades, the pharmaceutical industry has invested huge resources and efforts to look for new bioactive compounds as drug candidates. Two major approaches are adopted in the course of new drug discovery: screening microorganisms for bioactive natural products, or screening chemical compound libraries for synthesized compounds having therapeutic potential.

TABLE 1
Examples of biofunctional natural products, their sources and therapeutic
applications.

| Therapeutic Applications | Compound/Class | Producing Organism |
|---|---|---|
| Antibacterial agents used to | Streptomycin/Aminoglycoside | [symbol] |
| treat infections by Gram | Chloramphenicol | [symbol] |
| positive and Gram negative | Oxytetracycline/Polyketide | [symbol] |
| bacteria | Gentamicin/Aminoglycoside | [symbol] |
| | Thienamycin/β-Lactam | [symbol] |
| Antibacterial agents used to | Penicillins/β-Lactam | Penicillium spp |
| treat infections by Gram | Erythromycin/Macrolide | [symbol] |
| positive bacteria | Rifamycins/Ansamycin | Amycolatopsis mediterranei |
| | Vancomycin/Glygopeptide | [symbol] |
| | Daptomycin/lipopeptide | [symbol] |
| | Fusidic Acid | Acremonium fisidioides |
| Antifungal agents | Amphotericin B/Polyene macrolide | [symbol] |
| | Nystatin/Polyene macrolide | [symbol] |
| | Griseofulvin | Penicillium griseofulvum |
| | Pneumocandins/Lipopeptide | Glarea lozoyensis |
| Antitumour agents | Doxorubicin/anthracycline | [symbol] |
| | Bleomycins | [symbol] |
| | Actinomycin D | [symbol] |
| | Mitomycin C | [symbol] |
| | Streptozotocin | [symbol] |
| | Calicheamicins | [symbol] |
| | | [symbol] |
| Immunosuppressive agents | Cyclosporin A | Tolypocladium inflatum |
| | FK506 | [symbol] |
| | Rapamycin | [symbol] |
| | Mycophenolic Acid | Penicillium brevi-compactum |
| Agents useful in the treatment | Mevinolin/Statins | Aspergillus terreus |
| of cardiovascular, neurological | Lipstatin | [symbol] |
| and metabolic diseases | Acarbose | [symbol] |
| | Ergometrine/Ergot alkaloids | [symbol] |

*Drugs/Sources typed in bold are produces by actinomycetes.

2.2. The Diversity of Microorganisms from the Environment

It has been estimated that the pharmaceutical industry has screened on the order of 10,000,000 microbes for natural products over the past 50 years. Despite these efforts, only a very small portion of microbial species has been screened due to the tremendous diversity of microbes and the limitation of available screening techniques.

Recent statistical analysis has estimated bacterial diversity, based on meaningful differences in the 16S rRNA gene sequences of organisms (one method for estimating genotypic diversity), as 160 taxa per mL of ocean water and from 6,400 to 38,000 taxa per g of soil. Curtis et al., 2002, “Estimating prokaryotic diversity and its limits,” Proc. Nat. Acad. Sci. 99(16):10494-499. Accordingly, the entire bacterial diversity of the oceans may be on the order of 2×10⁶ different taxa and 10 m² soil may contain as many as 4×10⁶ different taxa. Id. Similarly, a recent study has estimated that Streptomyces, the largest antibiotic producing genus, is capable of producing on the order of 100,000 antimicrobial compounds, a mere fraction of the number discovered to date. Watve et al., 2001, “How many antibiotics are produced by the genus Streptomyces?,” Arch. Microbiol. (176):386-390. Effectively screening this microbial diversity for new molecules is further hampered by the constantly repeated discovery of known natural products produced by microorganisms, which are either the major populations in the environment, or are relatively easier to grow under available techniques of fermentation.

2.3 Efficient Drug Screening Systems Seeking Useful Natural Compounds Produced by Microorganisms.

An efficient drug screening system should have one or more of the following elements: (1) the whole procedure is fast and high throughput, which means that the system should be able to screen a sample with high diversity in a short period of time; (2) the screening outcome has low background, which means that the probability of obtaining novel compounds (unknown compounds or known compounds with novel functions) should be high; (3) the screening method is simple and sensitive; (4) the screening read-out is easy to detect.

2.3.1 Efficient and Ultra-High Throughput

As mentioned above, one problem that researchers encountered during the course of screening natural compounds from microorganisms is that the target microorganisms are usually in relatively low abundance in original samples, such as microorganism samples collected from soil. For example, actinomycetes, the major producers of bioactive natural products, constitutes only ˜1% of culturable microorganisms in soil samples. Consequently, other microorganisms in the sample may hinder the growth of actinomycetes and/or production of natural products from actinomycetes. An efficient drug discovering system should be able to screen a large number of microorganisms with high diversity in a short period of time and single out a specific species producing desired useful natural compounds efficiently, even though the targeted microbial species is a very small proportion of the whole sample. Unfortunately, the currently available drug screening procedures are not efficient enough to screen for microorganisms producing useful natural compounds with a satisfactory success rate when such microorganisms are among the less abundant species in the sample pool.

2.3.2. Prevent Rediscovery

Rediscovery, the identification of previously known antibiotics through tedious drug screening procedures, hinders the discovery of novel compounds and wastes resources. One approach to lower this background in drug screening is to use single or multiple antibiotic-resistant microorganisms as the test strains (termed screening strains in this invention) to screen natural product extracts for antibacterial activities. These single or multiple drug-resistant screening strains are either isolated clinically or constructed by random mutagenesis. Sugie et al have identified new antifungal compounds using clinically isolated multi-drug resistant bacterial strains. See, e.g., Sugie et al., 2002, “A novel antibiotic CJ-17,572 from a fungus, Pezicula sp.,” J. Antibiotics 55 (1):19-24; Sugie, et al., 2002, “CJ-21,058, a new SecA inhibitor isolated from a fungus.”, J. Antibiot. 55(1):25-9. In another study, a panel of four Gram-positive and two Gram-negative multi-drug resistant clinical strains was used to evaluate the antimicrobial activity of compounds produced by endophytic fungi. Pelaez et al., 1998, “Endophytic fungi from plants living on gypsum soils as a source of secondary metabolites with anti-microbial activity,” Mycol. Res. 102(6):755-761.

Others use randomly mutagenized bacterial or yeast strains for drug screening. For example, researchers have used UV mutagenesis to generate a C. albicans strain resistant to a broad range of lipopeptides and certain glycolipid inhibitors. Frost et al., 1997, “Characterization of a lipopeptide-resistant strain of Candida albicans,” Can. J. Microbiol. 43(2):122-128. The authors further proposed using the mutant strain in a dereplication assay to avoid the discovery of common, naturally-occurring lipopeptides during drug screening. In another study, Etienne and colleagues have established randomly mutagenized S. cerevisiae strains resistant to multiple polyene macrolides. These yeast strains are useful for the rapid detection of new antifungal compounds. Etienne et al. 1990, “A screening method for antifungal substances using Saccharomyces cerevisiae strains resistant to polyene macrolides,” J. Antibiotics. 43(2):199-206.

An alternative approach to avoid “rediscovery” is to select target-specific compounds so that known antibiotics, which function via different mechanisms, will not be selected. DeVito et al. constructed an array of Escherichia coli strains. Each E. coli strain expresses a low level of an essential gene product, which makes it hypersensitive to specific inhibitors of that gene product. DeVito et al., 2002, “An array of target-specific screening strains for antibacterial discovery,” Nature Biotechnol. 20:478-483. Screening these strains against a large chemical library permitted the identification of compounds with good inhibitory activity against more than one essential target. Other studies targeted inhibitors of cell wall synthesis. DeCenzo et al. had used an E. coli envA-strain carrying a plasmid based β-lactamase gene to screen compound libraries, natural product libraries, via detection of β-lactamase induction and identified inhibitors of peptidoglycan synthesis. DeCenzo et al., 2002, “Identification of compounds that inhibit late steps of peptidoglycan synthesis in bacteria,” J. Antibiotics, 55(3):288-295. Another screening system utilized two E. faecalis strains constructed to detect compounds that interfere with cell wall glycosylation. Sancheti et al., 1998, “Screening systems for detecting inhibitors of cell wall transglycosylation in Enterococcus,” J. Antibiotics, 51(6):471-479. Although the aforementioned drug screening systems work efficiently in reducing some background, they still permit rediscovery of many known compounds

2.3.3. Screening Methods and Screening Read-Outs

All efficient drug-screening systems should be simple and sensitive. In addition, the screening read-outs should be easy to detect. The commonly applied screening method is to monitor cell growth, a direct method to detect antimicrobial activities. Alternatively, reporter genes are used to detect the presence of agents regulating either a specific signaling pathway, or the expression of a specific gene product. For example, researchers have used a SecA-LacZ fusion reporter construct in E. coli to identify and analyze bacterial protein secretion inhibitors. Alskne et al., 2000, “Identification and analysis of bacterial protein secretion inhibitors utilizing a SecA-LacZ reporter fusion system,” Antimicrobial Agents and Chemotherapy 44(6):1418-1427.

2.4. Some Limitations of Available Drug Screening Systems and Some Advantages of the Present Invention

Unfortunately, none of the available screening systems provides sufficiently high throughput to exploit the magnitude of microbial diversity that still exists for discovery of novel, useful compounds, such as antibiotics. In fact, many pharmaceutical companies have abandoned the search for natural product therapeutics because of a lack of suitable processes and resources for screening and identifying novel natural compounds. In addition, despite the various efforts made to discover drugs from both synthetic and natural sources, there has been a paucity of new drugs. As a matter of fact, except for linezolid and Cubicin® (daptomycin for injection), the pharmaceutical industry has not provided new chemical classes of antibiotics to clinical practice for over 30 years.

### 3. SUMMARY OF THE INVENTION

The present invention features a genetically engineered microorganism that is useful for screening natural products for biological activity. In one aspect, the invention provides a genetically engineered microorganism having several genetically engineered traits that are useful for high throughput screening of microorganisms isolated from environmental sources. Examples of such genetically engineered traits include resistance to counterselection drugs, resistance to certain antibiotics, and auxotrophy, as described in further detail herein.

In a specific embodiment, a cell with two or more different drug resistance genes are artificially recombined into a chromosome of a cell. These drug resistance genes can be placed in different loci of the chromosome as desired. The number and type of drug resistance genes can be selected to fit a particular screening method or isolates. The genotype of the cell can be engineered or mutated to have particular phenotypes or genotypes, such as auxotrophy, increased or decreased cellular membrane permeability, sensitivity to toxins, reporter genes and promoter genes. The cells that are included in the invention are bacteria, fungi, mammalian cells, plant cells, and insect cells. The invention further teaches the various methods for making such cells.

In certain embodiments of the screening method of the invention, the cells taught herein can be used to screen for compounds having a desirable effect on the cell. The screening methods can screen against the natural products of whole cell microorganisms. In another aspect, the invention provides a method of screening microorganisms isolated from, for example, environmental sources or libraries. In particular, the method of the present invention allows screening for natural products produced in situ without extraction of metabolites or removal of the producing organism. For example, in one embodiment, the method of the invention may be used to screen for novel natural product antibiotics produced by actinomycetes. In addition, a method of the invention avoids re-discovery of known antibiotics and/or other natural product producing organisms that may interfere with discovery of novel, useful compounds. The methods can be used to accomplish ultra high throughput screening.

### 4. BRIEF DESCRIPTION OF THE DRAWINGS

FIG. 1: Overview flow chart illustrating the drug discovery procedure.

FIG. 2. The two-layer agar diffusion bioassay screening for cytotoxic agents using a human tumor cell line as the screening strain.

FIG. 3. Iterative process of introducing drug resistance genes into precursors of the screening strains.

FIG. 4. The distribution of the drug resistance genes and expression cassettes in the screening strain CM166.

### 5. DETAILED DESCRIPTION OF THE INVENTION

### 5.1. Definitions

The terms defined below shall have the meanings ascribed below, unless an alternate interpretation is otherwise required by context.

The term “artificial change in the genotype” refers to the alteration of a nucleic acid sequence using genetic engineering methods. For example, artificial change in the genotype includes site directed mutagenesis and artificial random mutagenesis caused by conditions imposed to mutate the cell. Artificial random mutagenesis includes, for example, growing a cell in the presence of increased UV radiation and treating the cell with mutagens. It excludes naturally occurring mutagenesis.

The term “artificial recombination” refers to the alteration of a nucleic acid sequence using genetic engineering methods. For example, artificial recombination includes site directed mutagenesis. However, it excludes random mutagenesis, even if artificially induced, and naturally occurring mutagenesis.

The term “auxotrophy” refers to the cell's dependence upon specific nutrients to survive. The specific nutrient has to be provided to the cell, as a result, the cell cannot grow under normal growth conditions. Mutation in a gene encoding products involved in the biosynthesis of an essential nutrient results in auxotrophy. Examples of useful auxotrophies in E. coli include, but are not limited to: (1) mutations of enzymes involved in vitamin biosynthetic pathways, such as the bioA or thiA locus (the resulting cell is dependent upon biotin or thiamin (vitamin B1), respectively); and (2) mutations in one or more of biosynthetic pathways of essential amino acids, for example, biosynthetic operons of Methionine (metA), and Valine/Isoleucine (ilvG), such that the resulting cell is dependent upon that particular amino acid.

The term “cell stress” refers to any physiological condition that negatively affects the normal growth of a cell. Some cell stress conditions include DNA damage, cell envelope damage, low or high temperatures, and hyper-osmotic stress.

The term “chromosome” refers to the stable DNA structure copied and transferred between generations of the cell during cellular division. As the cell divides, a chromosome is copied and a copy is transferred to each of the progeny cells. Absent unusual circumstances, the chromosome is stably replicated and transferred to each progeny cell in each of the successive rounds of cell division. A chromosome does not have to be native to the cell, an example being the yeast artificial chromosome, however, plasmids and transposons, which can be lost during cell division and are more easily transferred between cells outside of division (and for these and other reasons are therefore not stable), are not chromosomes.

The term “chromsomal locus” refers to a position on the chromosome.

The term “counter-selection drug” refers to an agent that selects against or inhibits microorganisms not of interest for screening but present in a large mixed pool containing the target microorganisms.

The terms “drug” and “therapeutic agent” are used interchangeably and refer to any bioactive agent or substance used in the prevention, diagnosis, alleviation, mitigation, treatment or cure of any disease. Such agents include active substances directed to specific physiological processes or systems, such as, but not limited to, diuretic, hepatic, pulmonary, vascular, muscular, cardiac or diabetic agents. Usually, such agents will modify the physiological performance of a target tissue or a type of cells in order to shift the physiological performance of the target tissue or cells towards a more homeostatic physiological state. Examples of preferred therapeutic agents include antimicrobial (e.g., antibacterial, antifungal and antiviral) agents, antitumor agents, immunosupressants, cardiovascular agents, and cytotoxic agents. See Table 1.

The term “drug-resistant genes” or “drug resistance genes” refer to DNA sequences encoding gene products, which make the cell containing such genes resistant to the corresponding drugs. These genes are usually identified from naturally isolated cells possessing a drug resistant or multi-drug resistant (“MDR”) phenotype. Transfer of specific drug-resistant genes into non-drug resistant cell strains may render the recipient strains resistant to the corresponding drugs. Some of the already identified, and possibly applicable in the present invention, drug resistance genes in the art are listed, but not limited to those listed, in Table 2.

The term “essential chromosomal locus” refers to a chromosomal locus that encodes a gene product necessary for cell growth, so that when the locus is mutated, deleted or disrupted, the cell is will not grow under normal growth conditions. Examples of essential chromosomal loci include loci that encode gene products involved in the biosynthesis of essential nutrients, so that when the genes are mutated, deleted or disrupted, the cell cannot grow under normal conditions.

The term “genetically engineered microorganism” refers to a microorganism that has been artificially modified to insert, substitute or delete one or more desired genetically determined traits. Similarly, “genetically engineered bacterium” refers to a bacterium that has been artificially modified to insert, substitute or delete one or more genetically determined traits; “genetically engineered fungus” refers to a fungus that has been artificially modified to insert, substitute or delete one or more genetically determined traits, and so on. Therefore, genetically engineered microorganisms are distinguishable from naturally occurring microorganisms that do not contain a foreign characteristic, such as an artificially introduced or mutated gene. Examples of some genetically engineered microorganisms useful in certain aspects of the invention show one or more of the following phenotypes: (1) resistance to one or more counterselection antimicrobial agents; (2) resistance to one or more additional antimicrobial agents; (3) auxotrophies; (4) any other genetically determined traits useful in the detection of desired microorganisms/natural compounds.

The term “herbicide” refers to compounds or other substances intended for killing plants or interrupting their normal growth. It may be a broadleaf, grass or brush killer.

The term “microorganism” as used herein includes bacteria, yeast, filamentous fungi and protozoans. Any microorganism producing useful natural compounds is the target microorganism in accordance with the present invention. Bacteria, including both Gram-positive (actinomycetes, in particular) and Gram-negative bacteria, are the preferred species. Yeasts, and particularly filamentous fungi, are also preferred sources for the screening of bioactive natural products. See Table 1.

The term “multi-drug resistant” or “MDR” phenotype refers to the cell's phenotype that such cell is resistant to one or more drugs or agents. Cells with an MDR phenotype are resistant to drugs, e.g., neomycin, streptomycin, trimethoprim, streptothricin, nalidixic acid, tetracycline, aminoglycosides, β-lactams, chloramphenicol, and apramycin. Cells possessing an MDR phenotype are either naturally isolated, or established by genetic engineering as described in the definition of “genetically engineered microorganisms.”

The term “non-essential chromosomal locus” refers to a chromosomal locus that encodes a gene product that is not necessary for the normal growth of the cell, so that when the locus is mutated, deleted or disrupted, the cell grows under normal growth condition. A non-essential locus can also be a locus that does not encode for any gene.

The term “pesticide” refers to a composition, chemical entity or mixture thereof that has the effect to prevent, destroy, repel or mitigate any pest which affects the viability of plants. It includes insecticides, rodenticides, nematicides, molluscicides, bactericides, fungicides, herbicides, algicides and the like.

The term “promoter gene” refers to the DNA sequence, often upstream of a gene that regulates the expression of the gene. In some cases, in response to physiological conditions, specific cellular proteins bind to the promoter gene to regulate the expression of the gene.

The term “reporter gene” refers to a gene sequence, whose phenotypic expression is easy to detect, that is often artificially introduced into a cell to monitor specific changes or conditions in the cell. Although not required, a reporter gene is often attached to a specific promoter gene to monitor changes in physiological conditions. Commonly used reporter genes include the lacZ, GFP and RFP genes.

### 5.2. Detailed Description

5.2.1. Introduction

There is a great clinical need for the development of ultra-high throughput screening strategies to aid the discovery of new drugs from both biological and chemical sources. The present invention addresses the need by providing solutions to the numerous problems and difficulties encountered in the process of screening natural products for bioactivity. The invention focuses on screening strains comprising bacteria, fungi and cell lines having desired genetic traits for new compound screening. In particular, these screening strains contain multiple drug resistance genes recombined into the chromosomes. Unlike randomly mutagenized and clinical isolates of multi-drug resistant strains, the screening strains of the invention can be predictable, stable, and well characterized, and can be designed to suit specific needs of the screens. The screening strains are particularly well suited to dereplicate known compounds in ultra-high throughput screening systems to discover novel and useful compounds. The invention further provides the method for constructing the screening strains.

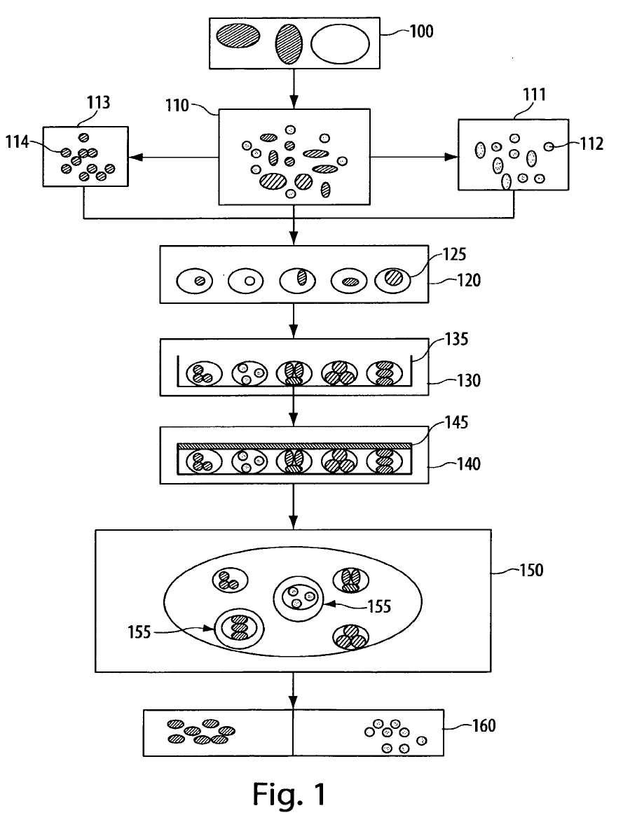

The present invention also teaches methods that can use the screening strains to deliver an ultra-high throughput screening. The method taught by the invention can, in certain embodiments, facilitate efficient discovery of novel bioactive natural products (such as antimicrobial compounds) produced by perhaps only a small percentage of organisms among a significantly large pool of microbes. In particular, in specific embodiments, the invention provides materials and methods that could enable screening in excess of 10,000,000 microbes for useful natural products in less than one year. The present invention also teaches methods for isolating and characterizing bioactive natural compounds from environmental samples. A screening procedure in accordance with the invention is schematically illustrated in FIG. 1 and various steps useful in the screening procedure of the invention are further described in detail in section 5.2.2 and 5.2.3.

These aforementioned screening methods for bioactive natural compounds and for microorganisms producing biologically active natural compounds are novel, and can be efficient and ultra-high-throughput. Certain embodiments of the invention permit a round of screening in about 3 months such that one might screen more than 10 million microorganism cultures per year. Under ordinary screening methods, a typical pharmaceutical company might screen 25,000 cultures per year. It would take 40 years for 12 large pharmaceutical companies to screen a total of 10 million microorganism cultures. The present inventions therefore could be used to significantly increase the screening rate for new natural compounds having therapeutic potential.

5.2.2. Screening Strains

In this section, desired properties of screening strains that can be used in the aforementioned screening methods and the process to construct these screening strains are taught.

5.2.2.1. Type of Screening Strains

The type of microorganisms used as screening strains should not be particularly limited and only need be suitable for use in a screening method of the inventions. In general, microorganisms, such as bacteria, yeast, mold, etc., can be used as screening strains as long as they can be cultured and genetically engineered, or can be selected from natural sources by the treatment of high-dose of antibiotics or mutagens. Bacteria, both Gram-positive and Gram-negative strains, are preferred screening strains in drug-screening methods introduced in this application. Many species of bacteria, especially E. coli, are well-established research systems in bioscience due to their well-characterized genomes and well-developed techniques to manipulate their genetic compositions. One skilled in the art is able to modify a bacterial strain so that it can serve a special purpose in drug-screening according to what type of compounds one intends to find or avoid, for example, multiple drug resistance genes can be introduced into a bacterial screening strain in order to avoid the detection of already known antibiotics. See details in section 5.2.2.2. Suitable bacterial species include, but are not limited to, Gram-positive cocci such as Staphylococcus aureus, Streptococcus pyogenes (group A), Streptococcus spp. (viridans group), Streptococcus agalactiae (group B), S. bovis, Streptococcus (anaerobic species), Streptococcus pneumoniae, and Enterococcus spp.; Gram-negative cocci such as Neisseria gonorrhoeae, Neisseria meningitidis, and Branhamella catarrhalis; Gram-positive bacilli such as Bacillus anthracis, Bacillus subtilis, Corynebacterium diphtheriae and Corynebacterium species which are diptheroids (aerobic and anerobic), Listeria monocytogenes, Clostridium tetani, Clostridium difficile, Streptomyces, Amycolatopsis, and Gram-negative bacilli including Burkholderia cepacia, Escherichia coli, Enterobacter species, Proteus mirabilis and other spp., Pseudomonas aeruginosa, Klebsiella pneumoniae, Salmonella, Shigella, Serratia, Campylobacter jejuni, and Acinetobacter. See Goodman and Gilman's Pharmacological Basis of Therapeutics, (8^{th} ed., 1990) Table 44-1, page 1024-1033, for additional microorganisms.

Fungal species also can serve as screening strains for the selection of antifungal agents, and natural compounds with other properties as well. Fungi screening strains resistant to currently available antifungal agents can be useful tools in the discovery of new classes of antifungal drugs. In addition, fungal cells are closer to human cells, compared to bacterial cells. Therefore, using fungi as test strains may allow the identification of potential drug candidates, which target human diseases not caused by bacterial or fungal infection, such as cancer or viral infection. See details in sections 5.3.4 and 5.3.7.

Multiple drug-resistant fungal strains can be established by natural selection against high doses of known antifungal drugs, such as polyenes (amphotericin B), pyrimidines analogues (5-FC), azoles (fluconazole, itraconazole, etc.), allylamines (terbinafine), morpholines (amorofine) or echinocandins (caspofungin). Many drug-resistant fungal strains can be obtained from various clinical sources. The methods of establishing such fungal strains by high-dose drug selection are also well known by skilled persons in the art. Etienne, et al, 1990. Drug resistant fungal strains also can be established by mutagen treatment followed by selecting a desired phenotype. Genetic engineering can be another way to construct fungal screening strains with desired genetic traits. Suitable yeast screening strains include, but are not limited to, Saccharomyces cerevisiae, Schizosacchromyces pombe, Phaffia rhodozyma, Kluyveromuces lactis, Pichia pastoris, Hansenula polymorpha, Yarrowia lipolytica, Candida albicans, C. tropicalis, C. lusitaniae or other Candida species, Torulopsis glabrata, Epidermophyton floccosum, Malassezia furfur (Pityropsporon orbiculare, or P. ovale), Cryptococcus neoformans, Aspergillus fumigatus, Zygomycetes (e.g., Rhizopus, Mucor), Paracoccidioides brasiliensis, Blastomyces dermatitides, Histoplasma capsulatum, Coccidioides immitis, and Sporothrix schenckii. Suitable filamentous fungi include, but are not limited to, Neurospora crassa, Aspergillus nidulans, Aspergillus niger, Aspergillus spp., Trichoderma spp., (such as T. rubrum and T. mentagrophytes), Microsporum canis and other M. spp., as well as Fusarium spp. See Goodman and Gilman's Pharmacological Basis of Therapeutics, (8^{th} ed., 1990) Table 44-1, page 1024-1033, for additional microorganisms.

In addition to microorganisms, adhesive cell lines, including, but not limited to, insect cells, plant cells and mammalian cells, can also be suitable screening strains. Mammalian cell lines, especially human cell lines, should be the optimal screening strains when one intends to look for natural compounds targeting human diseases that are not caused by either bacterial or fungal pathogens.

The foregoing microorganisms and cell lines can be available, for example, from the American Type Culture Collection. Methods to culture and genetically modify the aforementioned microorganisms and cell lines in vitro are well known in the art. See e.g. Sambrook, et al, “Molecular Cloning: A Laboratory Manual,” Cold Spring Harbor Press, 3d Ed. 2001; See also Ausubel, et al., “Current Protocols in Molecular Biology,” John Wilay & Sons, (updated online annually); Demain, et al., “Manual of Industrial Microbiology and Biotechnology,” ASM Press, 7^{th} Ed., 1999; Vinci, V. et al., “Handbook of Industrial Cell Culture Mammalian, Microbial, and Plant Cells,” Humana Press 2003 (particularly “Natural Products: Discovery and Screening” and “Genetic Engineering Solutions for Natural Products” pp. 107-170).

5.2.2.2. Genetic Traits of the Screening Strains:

Those skilled in the art will recognize from the teachings herein that the type of genetic traits of screening strains should depend largely on the types of natural products sought. For example, when new antibiotics are the screening targets, genes resistant to common antibiotics, such as tetracycline, neomycin, etc., should be preferred genetic traits of the screening strains so that these previously discovered antibiotics will not be detected in the course of drug-screening.

In general, the suitable screening strains will have one or more of the follow genetic traits: (1) drug-resistant genes against commonly known antibiotics; (2) genes conferring resistance to counter-selective agents, for example, the agents applied to enrich for actinomycetes; (3) genetic mutations that render the screening organism auxotrophic for specific nutrients not abundantly present in the environment, thus making these screening strains “biosafe”; (4) reporter fusion genes, such as lacZ gene fused with a selected promoter sequence; or (5) other desired genetic traits in accordance with the type of compounds sought, e.g. mutations aimed at increasing drug permeability. See following sections for details of these genetic traits.

5.2.2.2.a. Multiple Drug Resistances

Resistance to one or more common antimicrobials should be a preferred genetic trait of screening strains and the combination of drug resistance genes that a desired screening strain possesses should be in accordance with the type of natural compounds sought. For example, when one intends to screen natural products of actinomycetes, it may be preferable to use a screening strain possessing genes conferring resistance to known antibiotics that are produced by actinomycetes, in order to avoid rediscovery. Depending on the need, screening strains may be created to have any number of drug resistances. For example, a screening strain may contain two or more drug resistances, e.g. three, four, ten, fifteen, twenty, or all known drug resistance genes for that organism. Examples of drug resistance genes, which are introduced into screening strains in different combinations, are listed in Table 2.

Resistance to antifungal drugs is widely observed in the medical community. Studies on the genetic basis of antifungal drug resistance have identified genes related to antifungal drug resistance. In the case of Candida, mutations of the Candida drug resistance (CDR) gene family members, such as ATP-binding cassette (ABC) transporter superfamily and MDR1, cause drug-resistance to azole and polyene. Ergosterol, the major component of fungal membranes, is the target of polyene antibiotics and the ergosterol biosynthetic pathway is the target of azole derivatives. Therefore, mutations of genes involved in ergosterol biosynthesis, or overexpression of these genes, e.g. ERG 6, ERG11, all can cause drug resistance to antifungal agents of the polyene and azole families. In other fungal species, mutation in CYP52A leads to itraconazole resistance in Aspergillus fumigatus. Introduction of the aforementioned gene mutations can result in fungal strains possessing multiple drug resistances. Methods for genetic modification of fungi, especially yeast, are well-known in the art. Demain & Davies, 1999. Vectors such as YAC, Ycp, Yep, YIp and YRp have been developed for non-Saccharomyces yeasts and 42 types of genes derived from different sources have been used as genetic markers monitoring genetic engineering of fungi. Wang et al., 2001, “Transformation system of non-Saccharomyces yeasts,” Grit. Rev. Biotechnol., 21(3):177-218. Methods for genetic engineering of non-Saccharomyces also have been well-established, for example, a spheroplast-mediated method, an alkaline iron treatment method, electroporation, trans-kindom conjugation and biolistics. Id. The biolistic approach can also be adopted to genetically manipulate fungi strains which are difficult to culture or whose protoplasts are difficult to obtain. Harrier & Millam, 2001, “Biolistic transformation of arbuscular mycorrhizal fungi, progresses and perspectives,” Mol. Biotechnol. 18(1):25-33.

Resistance to counter-selective agents should be another preferred genetic trait of screening strains. Counterselective drugs may be used to enrich for a particular population of microorganisms. For example, when natural products produced by actinomycetes are the screening targets, drugs such as trimethoprim (Tmp) and nalidixic acid (NaI) may be used to enrich for the actinomycete population from environmental samples by reducing or deselecting other microorganism populations, such as Gram-negative and other Gram-positive soil bacteria. See Hayakawa, M., Takeuchi, T. and Yamazaki, T. 1996, “Combined use of trimethoprim and nalidixic acid for the selective isolation and enumeration of actinomycetes from soil,” Actinomycetol. 10(2):80-90. Thus, strains bearing resistance against counterselective drugs (Trm and/or NaI) may be the preferred test strains in screening natural products produced by actinomycetes. Trm and NaI resistant genes have been well-characterized in the art. Exemplary resistance determinants for Trm are dihydrofolate reductase genes (such as that encoded on Tn7; Genbank accession # BAB 12602). NaI-resistance is encoded, for example, by several different gyrA (Genbank accession # X06373) alleles. Also see Table 2. Application of different counter-selective agents will lead to the isolation of different targeted sub-population of microorganisms.

Multi-drug resistant cells can be created by random mutagenesis or isolated from clinical patients. However, cells with known drug resistance genes stably recombined into specific loci in the chromosomes have many potential advantages. Unlike clinical isolates or randomly mutagenized cells, which may not be well characterized, these engineered strains should be predictable in their genotypes, growth requirements and drug resistance mechanisms. Predictability means fewer false leads and less effort to characterize the results of the screen. Furthermore, depending on the types of compounds sought, the screening strains may need to have a large number of drug resistances, and maintain these resistances stably through the screening process. Clinical isolates often carry their drug resistance genes on plasmids or transposons, which are less stable. Additionally, clinical isolates or randomly mutagenized strains are unlikely to occur having the exact drug resistances needed. Having all the desired drug resistances stably in one single screening strain may be important for the ability to screen in a sufficiently high-throughput manner to discover novel compounds that have eluded ordinary methods.

TABLE 2
Examples of drug resistance genes

| Drug resistance | gene | source | supplier | GenBank # |
|---|---|---|---|---|
| Ampicillin | pbp5^{#} | E. faecalis | Available in almost all | X78425 |
| | blaZ | S. aureus plasmid | research labs | AY373761 |
| Aminoglycosides** | aph(2″)-Ib | E. faecium | Joseph Chow†† | AF207840 |
| | aac(6′)-Im | | | AF337947 |
| Apramycin | aac(3′)-IV | pStreptoBACV | Cubist | AJ414670 |
| Bleomycin | ble | Tn5 | E. coli Genetic Stock | X01702 |
| | | | Center (#7382) | |
| β-lactams | TEM-1 bla | PQEΔNde2 | Cubist^{##} | X54604 |
| Chloramphenicol | cat | pKD3 | E. coli Genetic Stock | AY048742 |
| | | | Center (#7631) | |
| Nalidixic acid* | nalA37 | UB1005 | E. coli Genetic Stock | D90854 |
| | | | Center (#7138) | |
| Neomycin | neo | pSU2007 | F. de la Cruz††† | V00618 |
| Spectinomycin | aadA1 | Tn7† | DSMZ 3872 (colE1::Tn7 | AP002527 |
| | | | plasmid) | |
| Streptomycin | aadA1 | Tn7† | DSMZ 3872 (colE1::Tn7 | AP002527 |
| | | | plasmid) | |
| Streptomycin | rpsL150 | MC4100 | E. coli Genetic Stock | AF312716 |
| | | | Center (#6152) | |
| Streptothricin | sat | Tn7 | DSMZ 3872 (colE1::Tn7 | AP002527 |
| | | | plasmid) | |
| Tetracycline | metE::tetA | CAG18491 | E. coli Genetic Stock | J01830 |
| | | | Center (#7464) | |
| Trimethoprim* | dfrB2 | pSU2007 | F. de la Cruz††† | X01702 |
| Trimethoprim | DhfrI | Tn7† | DSMZ 3872 (colE1::Tn7 | AP002527 |
| | | | plasmid) | |
| Vancomycin | vanA | Tn1546 | Arthur et al.*** | M97297 |
| Antifungal | Genes | Species | References*** | Genbank # |
| itraconazole | CYP51A | A. fumigatus | Mellado, et al., 2001 | AF338659 |
| azoles | ERG11 | C. albicans | | AB071960 |
| amphotericin | ERG11 | C. neoformans | Rodero, et al., 2003 | AY265353 |
| fluconazole | ERG 6 | C. lusitaniae | Young, et al., 2003 | AY179503 |

*Counter-selective agents for the enrichment of actinomycetes.
**Examples of covered antibiotics
***Arthur at al., 1993, J Bacteriol. 175 (1): 117-127; Mellado, et al., 2001, J. Clin. Microbiol. 39(7): 2431-2438; Rodero, et al., 2003, Antimicrob. Agents Chemother. 47(11): 3653-3656; Young, et al., 2003, Antimicrob. Agents Chemother. 47(9): 2717-2724.
^{#}Overproduction of pbp5 results in resitance to β-lactoms. Fontana et al., 1983, J. Bacteriol., 155(3): 1343-5- and Arthur et al., 1993, J Bacteriol 175(1): 117-127.

Microbiol. 39(7):2431-2438; Rodero, et al., 2003, Antimicrob. Agents Chemother. 47(11):3653-3656; Young, et al., 2003, Antimicrob. Agents Chemother. 47(9):2717-2724.

# Overproduction of pbp5 results in resistance to β-lactams. Fontana et al., 1983, J. Bacteriol., 155(3): 1343-50 and Arthur et al., 1993, J Bacteriol 175(1): 117-127.

## Modified from pQE60 (Qiagen, Inc.): Created an Nde1 restriction site at position 114 by fusion with blunt ended NcoI site “ccatatg” and eliminated the Nde1 site at position 1369 by insertion of nucleotide in restriction site “cattatg”.

† The drug resistance genes associated with Tn7 are contiguous and introduced as a modified cassette in which the cryptic sat promoter region is replaced by a synthetic promoter (Ptrc; Amman et al., 1988, Gene 30; 69(2):301-15). The modified cassette is termed “miniTn7+”.

†† Kao et al., (2000) Antimicrob. Agents Chemother., 44(10): 2876-2879.

††† Martinez and de la Cruz, 1988, Mol Gen Genet., 211(2):320-5. Plasmid pSU2007 is a sulfonamide-sensitive derivative of R388 that contains the Tn5 neo gene (F. de la Cruz, personal communication).

5.2.2.2.b. Auxotrophies

Auxotrophy, the microorganism's dependence upon specific nutrients to survive, can be another preferred genetic trait in order to generate “biosafe” screening strains. Auxotrophic strains usually cannot survive outside of the laboratory where the required metabolite(s) are not readily available. As discussed in section 5.2.2.2.a above, screening strains used in the present inventions can be resistant to multiple commonly used antibiotics. It would be desirable to introduce an auxotrophic phenotype into such screening strains so that they are unable to survive outside of laboratory conditions and cause infections. The type of auxotrophic phenotype of a screening strain should have no particular preference and one or more auxotrophies may be chosen. Examples of useful auxotrophies include, but are not limited to: (1) mutations of enzymes involved in vitamin biosynthetic pathways, such as the bioA or thiA locus (the resulting strain is dependent upon biotin or thiamin (vitamin B1), respectively); and (2) mutations in one or more of biosynthetic pathways of essential amino acids, for example, biosynthetic operons of Methionine (metA), and Valine/Isoleucine (ilvG), such that the resulting strains are dependent upon that particular amino acid. Other mutations that could result in a strain unable to grow outside of the laboratory environment, include mutations in genes involved in uptake or utilization of specific carbon sources, e.g. ora (arabinose utilization), lac (lactose utilization). See Table 6.

5.2.2.2.c. Genetic Traits that Allows the Screening of Natural Compounds Acting Through Specific Mechanisms.

In many cases, one would desire to screen drugs that act through a specific mechanism, e.g. act against specific cellular targets, inhibit certain biopathways, or induce special cell responses. This strategy may have special preference when one intends to screen drugs that can act through a mechanism involved in a particular human or animal disease. For example, one skilled in the art can screen compounds that inhibit DNA synthesis or cell division as potential anti-cancer agents. Introduction of reporter fusion genes can be one of the many approaches to detect natural compounds acting through certain mechanisms. The expression of the reporter genes can be controlled by selected promoters, which will be turned on only under certain physiological conditions, such as cytotoxicity, DNA damage or other cell stress responses, etc. For example, a screening strain containing a lacZ reporter gene fused with a promoter sequence, which is sensitive to DNA damage, can be used to screen DNA damaging agents by detecting the expression of the lacZ reporter gene. This above screening strain can serve as a powerful tool in the discovery of anti-tumor agents since tumor cells are usually super-sensitive to DNA damaging drugs. Examples of stress-responsive promoters are listed in Table 3. These promoters can be used to construct the reporter fusion genes aforementioned. Other promoters sensitive to certain physiological conditions also can be used in the present inventions. For example, the vanH promoter can be used to select agents that inhibit cell wall transglycosylation. Mani, et al., 1998, “Screening systems for detecting inhibitors of cell wall transglycosylation in Enterococcus: Cell wall transglycosylation inhibitors in Enterococcus,” J. Antiobiot. 51(5):471-9. The type of promoter chosen should depend on the type of compounds sought and the type of screening strains selected in the course of drug screening. For example, bacterial SOS-response promoters can be selected to screen natural compounds that induce cell stress in bacteria.

TABLE 3
Stress-response Promoters Suitable for the Construction
of Reporter Fusion Genes

| Stress Sensor | Promoter Region | GenBank # |
|---|---|---|
| DNA damage | recN | Y00357 |
| Inhibition of translation (C group) | cspA | M30139 |
| Inhibition of translation (H group) | ibp | M94104 |
| Cell envelop damage | rpoH (P3) | M20668 |

* Shapiro et al., 2002, “Stress-based identification of antibacterial agents: second-generation Escherichia coli reporter strains and optimization of detection,”, Antimicrob. Agents Chemother. 46(8): 2490-2497.

Commonly used reporter genes, such as lacZ, GFP, RFP, lux, etc. all can be used to construct screening strains. LacZ may be a preferred reporter gene because the expression of lacZ reporter gene can be detected by in situ staining. Thus using lacZ reporter gene may allow the detection of active compounds that can induce the expression of the reporter gene but have poor ability to inhibit cell growth. A reporter gene can be fused with desired promoters, such as those listed in Table 3. When lacZ is chosen as the reporter gene, its promoter region (upstream region of lacZ, including lad) can be replaced with the selected promoter. The selected promoter can be amplified from its corresponding sources by PCR and insert into the upstream region of the lacZ. By doing so, the expression of lacZ reporter gene can be controlled by the selected promoter. The expression of the lacZ gene should be completely dependent on the selected promoter. Reporter fusion genes can be introduced into screening strains individually, or combined with other desired genetic traits. Strains containing reporter fusion genes can also be used as the test strains in secondary screening phases to confirm or build on initial results.

An alternative approach to screen for agents inducing cell stress responses, for example, DNA damaging agents, is to construct a screening strain that is ultra-sensitive to stress responses. One can generate such a screening strain by nullifying the function of one or more components of the bacterial SOS response system, such as making non-functional mutation of the recA gene or rendering the cells more permeable to drugs. See details in section 5.3.3.c and d.

5.2.2.2.d. Other Desired Genetic Traits of Screening Strains

Screening strains bearing these mutations may either have disrupted integrity of their cell envelope or have less efficient export systems, thus being hyper-sensitive to certain types of antibiotics. In detail, by combining mutations in TolC (tolC5::metC::tetA, CGSC #6098) with the multi-drug resistant genes, one is able to generate a strain likely to be more susceptible to antibacterial and cytotoxic compounds. Vaara & Nurminen, 1999, “Outer membrane permeability barrier in Escherichia coli mutants that are defective in the late acyltransferases of lipid A biosynthesis,” Antimicro. Agents Chemother. 43(6):1459; Sulavik, et al., 2001, “Antibiotic susceptibility profiles of Escherichia coli. strains lacking multidrug efflux pump genes,” Antimicro. Agents Chemother. 45(4):1126-1136. One can genetically link the tolC mutant alleles with drug resistance markers or other desired genetic traits and can then transfer the whole cassette into the recipient strains to construct screening strains in accordance with the type of natural compounds one intends to seek.

5.2.2.2.e. Genetic Traits of Screening Strains for the Purpose of Screening Antiviral Compounds

It is well-known in the art that the entry of virus into its host cells depends on the anchoring of viral envelope proteins to the host cell surface receptors/co-receptors. Fields, et al., 2001, “Virology,” Lippincott Williams & Wilkins, ed 3rd. Compounds that can block this anchoring event have the potential to prevent viral infection; Schols, 2004, “HIV co-receptors as targets for antiviral therapy,” Curr. Top. Med. Chem., 4(9):883-93. For example, HIV virus infects human T lymphocytes via the anchoring of its envelop protein, gp120, to T cell surface receptor CD4, as well as other co-receptors. Ruibal-Ares et al., 2004, “HIV-1 infection and chemokine receptor modulation,” Curr. HIV Res. 2(1):39-50. Matthews et al., have developed the first drug (Fuzeon) to inhibit the entry of HIV-1 into host cells. Matthews et al., 2004, “Enfuviride: the first therapy to inhibit the entry of HIV-1 into host CD4 lymphocytes,” Nat. Rev. Drug Discov. 3(3):215-25. Accordingly, the present inventions teach a method for selecting antiviral compounds which can disrupt the association between viral envelope proteins and host cell surface receptors/co-receptors.

A strategy similar to the yeast two-hybrid system can be adopted in the method for the selection of antiviral agents as described above. Karimova et al., 2002, “Two-hybrid systems and their usage in infection biology,” Int. J. Med. Microbiol. 292(1):17-25. In general, three additional genetic traits can be introduced into the screening strains, which can be bacterial, fungal or mammalian cell lines. Fungal strains (S. cerevisiae) and mammalian cell lines may be the preferred screening strains because they can provide close-to-natural post-translational modifications of proteins and such modifications usually are essential in protein-protein interactions. The first genetic trait can be a reporter gene fused with a transcriptional element in a way that the expression of the reporter gene is dependent upon the presence of a transcriptional factor, which can specifically bind to the aforementioned transcriptional element and drive the reporter gene expression. The transcriptional factors have two functional domains, the activation domain (AD) and the DNA binding domain (DBD). It is functional in terms of driving the reporter gene expression only when the AD and DBD domains are associated. The second genetic trait can be an expression cassette of an AD-vEP (viral-envelope-protein) fusion protein. The third genetic trait can be an expression cassette for a DBD-cSR (cell-surface-receptor) fusion protein. The vEP fragment can switch with the cSR fragment to form different fusion proteins as long as the AD and the DBD fragments are located at different fusion proteins. The interaction of the vEP and the cSR may result in the association of AD and DBD domains; this subsequently turns on the reporter gene. The presence of compounds that can disrupt the said interaction destroys the association of AD and DBD, resulting in the turning-off of the reporter gene. Commonly used reporter genes, such as lacZ, GFP, RFP, etc. all can be used as reporter genes in this screening method. In cases when in situ staining is a preferred way to detect reporter gene expression, one may choose lacZ as the reporter gene. The presence of the potential antiviral natural compounds can be detected when the reporter gene is silenced. See details in section 5.3.8.

Variable screening strains, each being designed for the purpose of screening a certain type of microorganisms producing natural bioactive compounds, have been established. These screening strains bear different combinations of genetic traits as discussed above, such as drug resistances, auxotrophies, reporter fusion genes and other desired genetic traits, in accordance with the type of compounds sought. Examples of the screening strains established are shown in Table 4. Drug-screening results have been achieved using these screening strains. Compounds identified using Ec1063, DR1212 and CM166 as screening strains are also listed in Table 4. Drugs to which the screening strain is resistant will not be detected using drug-screening methods taught by the present inventions. Thus the use of multi-drug resistant screening strains can effectively avoid rediscovery of these antibiotics. Additionally, when a screening strain is found to be sensitive to new, commonly known antibiotics during screening, one may further incorporate drug resistance genes corresponding to those particular antibiotics during the construction of further generations of screening strains. Doing so can significantly reduce rediscovery and increase the chances of obtaining novel compounds.

TABLE 4
Exemplary E coli screening strains constructed for drug screening

| Screening | | | Other genetic | Compounds |
|---|---|---|---|---|
| Strains | Drug-resistance | Auxotrophy | Traits | Detected |
| Ec1063 | nystatin, cycloheximide | | | streptothricins |
| | nalidixic acid, trimethoprim | | | actinomycins |
| | aminoglycoside | | | chloramphenicol |
| DR1212 | chloramphenicol, streptomycin | biotin, | recA | chrysomycins |
| | tetracycline, spectomycin | methionine | | streptonigrin |
| | streptothricins, trimethoprim | | | daunomycins |
| | aminoglycosides, beta-lactamase | | | chromomycins |
| | neomycin | | | |
| CM166 | aminoglycosides, apramycin | biotin, curli, | | Thiolutin |
| | beta-lactamase, chloramphenicol | methionine | | aureothricin |
| | nalidixic acid, neomycin | | | lydimycin |
| | spectinomycin, streptomycin | | | albomycin |
| | streptothricin, tetracycline | | | streptavidin |
| | trimethoprim, bleomycin | | | stravidin-like |
| CM400 | aminoglycosides, apramycin | curli | | amicetin |
| | beta-lactamase, chloramphenicol | methionine | | griseolutein |
| | nalidixic acid, neomycin | | | netropsin |
| | spectinomycin, streptomycin | | | nocardamine |
| | streptothricin, tetracycline | | | |
| | trimethoprim, rifamycin | | | |
| | albomycin, bleomycin | | | |
| CM435 | aminoglycosides, apramycin | curli | tolC | aureothin |
| | beta-lactamase, chloramphenicol | methionine | fhuA | borrelidin |
| | nalidixic acid, neomycin | | | bottromycin |
| | spectinomycin, streptomycin | | | globomycin |
| | streptothricin, tetracycline | | | nodusmicin |
| | trimethoprim, rifamycin | | | |
| | albomycin, bleomycin | | | |
| CM242 | streptomycin, tetracycline | Methionine, | P_{recN}-lacZ | to be identified |
| | spectinomycin, streptothricin | biotin, curli, | | |
| | trimethoprin, apramycin | | | |
| | aminoglycosides, bleomycin | | | |
| CM191 | neomycin, beta-lactamase | Methionine, | P_{sauA}-lacZ | to be identified |
| | streptomycin, tetracycline | biotin, curli | | |
| | spectinomycin, streptothricin | | | |
| | trimethoprin, apramycin | | | |
| | aminoglycosides, bleomycin | | | |
| | neomycin, beta-lactamase | | | |

5.2.3 General Techniques of the Ultra-High Throughput Drug-Screening Methods

The present inventions teach methods for high throughput screening of microorganisms producing natural products with desired bioactivities (e.g., therapeutic agents, herbicides, pesticides, etc.). As discussed in section 5.2.2 in detail, selected or genetically engineered microorganisms and cell lines can be used as test strains in the introduced drug screening methods.

5.2.3.1. Sample Sources and Enrichment Procedures:

Environmental sources are rich in microorganisms. For example, soil can contain up to 10⁹ cultivatablebacteria per gram of material from which 10⁶ to 10⁷ actinomycete spores can be present. Soils from different ecological and geographical locations may contain different populations, including numbers of actinomycetes, and other microorganisms. Horan, A. 1994, “Aerobic actinomycetes: a continuing source of novel natural products. In: The discovery of natural products with therapeutic potential.” Ed. Gullo, V. P. Butterworth-Heinemann, USA. Therefore, environmental samples, including soil and water can be the preferred sources for drug screening. A collection of diverse environmental samples can be pooled (item 100, FIG. 1) and the microorganisms extracted to a liquid form for storage and subsequent drug screening. The potential value of pooling sources of microorganisms can be exemplified by searching for producers of antibiotics rarely found in natural product drug discovery programs over the last 40 years. Examples of such antibiotics include erythromycin produced by Saccharopolyspora erythraea and vancomycin produced by Amycolatopsis orientalis. It is estimated that the frequency of the occurrence of these genera in soil is approximately 0.1%. By pooling soils and applying stringent antibiotic treatments that deselect against organisms that do not possess resistance mechanisms for these antibiotics, a few organisms can be isolated that may potentially contain the biosynthetic pathway for the antibiotic or be producers of novel compounds related to the antibiotic. The chances of finding such rare and potentially novel microorganisms may be improved by increasing the diversity of organisms to which the stringent antibiotic treatments are applied, such as pooling a number of diverse soils.

Microorganisms may be extracted from environmental samples, e.g. soil samples, using a variety of methods, which are known by skilled persons in the art (item 110, FIG. 1). Lindahl, V. and Bakken, L. R. 1995, “Evaluation of methods for extraction of bacteria from soil,” FEMS Microbiology Ecology, 16(2):135-142. Preparation of samples for use in methods of the inventions should depend upon the type of samples one will start with. For convenience, soil samples may be dried without sieving and macerated prior to extraction. Microorganisms extracted from environmental samples can be subject to encapsulation and macrodroplet assay as described in sections 5.2.3.2, 5.2.3.3 and 5.2.3.4, below, see item 120 in FIG. 1. Optionally, variable preselection methods can be adopted to enrich for interesting microorganisms. As mentioned above, actinomycetes are in most cases the preferred microbial source for antimicrobial screening. Over 10,000 presently known bioactive natural compounds have been isolated from various species of actinomycetes (7,600 from Streptomyces), thus actinomycetes are obviously the preferred target microorganisms in accordance with the present invention. See Berdy, 2005, “Bioactive microbial metabolites,” J. Antibiot. 58(1): 1-26, Table 4.

Therefore, it is preferable to enrich for actinomycetes from microorganisms extracted from environmental samples. See item 111, FIG. 1.

A two-step procedure can be applied to enrich for actinomycetes and to reduce populations of fungi and other non-actinomycete bacteria from environmental samples. The first step in this enrichment process should be a physical treatment, which can be achieved by drying the soil samples followed by generation of a bacterial pellet through a dispersion and then differential centrifugation technique to bias for the recovery of antinomycete spores. Herron, P. R. and Wellington, E. M. H. 1990, “New method for extraction of streptomycete spores from soil and application to the study of lysogeny in sterile amended and nonsterile soil,” Appl. Environ. Microbiol., 56(5):1406-1412. Also see FIG. 1, item 111. In detail, soil samples can be dried for up to seven days in a Class II safety cabinet to reduce the number of gram-negative bacteria, which can be followed by sieving or maceration of the soil to form fine particles suitable for extraction. Additional treatments on air-dried soil can be performed to alter the microbial population of the soil, such as reducing the number of non-actinomycete bacteria or biasing the population of actinomycetes to particular groups. Soil particles can then be extracted using a dispersal agent (0.1% cholic acid in 2.5% PEG8000, preferably), together with chelex 100 resin and shaken at 5° C. for around 2 hours to dissociate bacterial cells and spores from soil particles. The slurry is then centrifuged at 2250 rpm for 1 minute and the resulting supernatant collected. The supernatant is then pooled and centrifuged at 3000 rpm for 30 minutes to generate a bacterial pellet, which is retained, and a supernatant, which is discarded. The bacterial pellet can be resuspended in cryoprotectant and stored in multiple aliquots at −135° C. prior to screening. The second step of this enrichment process is the growth of the above soil sample extract in the presence of counter selective antibiotics, such as nalidixic acid (NaI) and trimethoprim (Trm, Hayakawa, 2000), which can effectively suppress or kill non-actinomycete bacteria. Cycloheximide and nystatin can also be introduced to reduce or eliminate fungi. Item 112 in FIG. 1 represents actinomycetes obtained after the enrichment process.

The invention also teaches methods designed to enrich for non-actinomycete microorganisms. See FIG. 1, item 113. In addition to antibiotic deselection, chemical or physical treatments also can be applied to obtain microorganisms of interest and the methods adopted should depend on the type of microorganisms or natural products one intends to isolate. Samples may go through chemical or physical treatments, such as heat treatment, variable media conditions (pH and/or salt concentration), incubation at different temperatures, etc., to enrich for specific genera/species of microorganisms. For example, soil samples can be treated by incubation at 30-35° C. for 2 to 3 hours in a germination medium followed by heating them at 65° C. for 10 minutes (“minor-shifted isolation”) in order to enrich for the minor population of Bacillus, e.g. B. polymyxa, B. pumilus, B. licheniformis and B. coagulans. Wakisaka & Koizumi, 1982, “An enrichment isolation procedure for minor Bacillus population,” J. Antibiot. 35(4):450-7. An alternative method discussed in Wood & Casida, 1972, can also be used to enrich sporangial subgroup II Bacillus species. “Soil enrichment for the isolation of sporangial subgroup II Bacillus species, and observations concerning a coil-forming member of this group,” Can. J. Microbiol. 18(7):1031-8. Methods for the enrichment for thermophiles using specific media and temperature can be found in A. Bull, 2004, “Microbial diversity and bioprospecting,” ASM Press, ch8:83. Item 114 in FIG. 1 represents the enriched microorganisms genera/species.

Microorganisms extracted from environmental samples, either with or without an enrichment procedure, can be serially diluted in phosphate-buffered saline (“PBS”) or other suitable buffer and an assessment should be made in nutrient media containing aforementioned counter-selective antibiotics to determine the colony forming units (cfus) of actinomycetes and other non-filamentous bacteria of the final extracts.

5.2.3.2. Encapsulation

The next step (FIG. 1, item 120) is to isolate and grow individual microorganisms contained in the above extracts. In a preferred embodiment termed “macrodroplet (MD) encapsulation”, a single cell or spore can be entrapped in a macrodroplet, made from a cross-linked alginate matrix containing fermentation medium with added counter-selective agents as described in section 5.2.2.1. The macrodroplet can be approximately 8 μl (although any size are usable), and represents a porous microenvironment containing a pure microbial culture that can be readily handled and fermented and can produce secondary metabolites (item 125 in FIG. 1). In detail, an aliquot of the bacterial suspension extracted from the soil should be diluted in 10% glycerol to generate an inoculum of the required density. This adjusted suspension is then mixed with nutrient medium, counter-selective agents (for example, Nalidixic acid 30 μg/mL, Trimethoprim 40 μg/mL, Nystatin 50 μg/mL, Cycloheximide 50 μg/mL) to inhibit growth of fungi and many non-actinomycete bacteria, and 1.4% sodium alginate. The resultant mixture is processed through an Inotech Encapsulator® Research device, or other suitable device, to produce gel beads (called macrodroplets, see item 125 in FIG. 1) by the formation of droplets of liquid from a fluid stream. The droplets can solidify into a gel as they come into contact with a 135 mM aqueous calcium chloride bath (curing), also containing nutrient medium and the counter-selective agents. Alternatively, the bacterial suspension can be mixed with the alginate solution and combined with the nutrient medium and counter-selective agents in the calcium chloride bath. After a defined hardening time the macrodroplets are washed in nutrient medium and counter-selective agents (for example, those aforementioned) to remove excess calcium chloride and to stop the hardening process. Using this procedure, hundreds of thousands of microorganisms can be encapsulated individually in these beads, which serve as miniature fermentation vessels. See details in sections 5.2.3.3, and 5.2.3.4. Also see FIG. 1, item 120.

Different media can be used with the alginate solution to encapsulate the microorganisms in order to select for the growth of the selected microorganisms, and/or the production of compounds by these microorganisms. This may be further varied by the inclusion of different antibiotics (for example, lincomycin, rifampicin, spectinomycin, erythromycin) or other chemicals (for example, bile salts). Different selection methods can be used to favor the growth of different sub-group of the microbial population. In the process of encapsulation, the concentration of the bacterial suspension can be adjusted according to the colony forming units under the culturing conditions applied.

The density of the microorganism suspension can also be adjusted in accordance with the desired number of microorganism cells or spores per gel bead. Routinely the density of microorganisms should be adjusted so that each gel bead contains, on the average, at least one microorganism colony. Alternatively said density can be adjusted such that one gel bead contains multiple microorganism colonies in order to capture compounds of potential therapeutic value produced as a result of the interaction of more than one microorganism.

Macrodroplets (containing microorganisms) can be suspended in the wash medium, and then spread in a 25×25 cm bioassay plate (up to 5,000 particles per plate). See item 130, FIG. 1. The growing medium should have multiple antibiotics and the types of antibiotics should depend upon the drug-resistant phenotype of the screening strain selected. Excess fluid should be removed and the macrodroplets should be effectively separated from each other. The macrodroplets can subsequently be incubated under controlled culturing conditions to allow the growth of the included microorganisms and the production of secondary metabolites such as antimicrobial agents. See item 130 in FIG. 1. If actinomycetes are the desired microorganism species, macrodroplets should be incubated at 28° C. for 5-10 days, but most often for 7 days, to expand the number of actinomycete cells included in the MD. Other culturing conditions, such as different medium composition, different culturing temperature, etc. can be applied when microorganisms other than actinomycetes are targeted. For example, E. coli and some other bacteria can be cultured at 35-37° C., while yeast and other fungi can be cultured at 25-30° C. During the microfermentation process described above, secondary metabolites are produced, secreted and diffuse within the medium matrix contained within the macrodroplet. These can subsequently be detected by the macrodroplet bioassay described below (FIG. 1, item 140).

5.2.3.3. Macrodroplet Screening Assay:

After the period of incubation as described above, macrodroplets can be screened for biological activities by a unique technique termed macrodroplet screening assay. The biological activities of compounds secreted by the encapsulated microorganisms and contained within the macrodroplet beads can be detected by, for example, cell growth inhibition. In general, screening strains should first be cultured to a certain cell density (proper OD reads as described in section 5.3.3.) in liquid medium and then plated in semi-solid medium matrix (0.8%), which overlays the macrodroplet beads (see FIG. 1, item 140. Item 145 of FIG. 1 represents the top layer of semi-solid medium matrix containing a selected screening strain). After being incubated overnight, the screening strain can grow throughout the matrix, except in areas where macrodroplet beads release metabolites, which prevent the growth of the screening strain. The inhibition of cell growth may result in zones of inhibition around these beads (see item 155, FIG. 1), which usually can be detected visually. See FIG. 1, item 150.

Optionally DNA can be added to the nutrient medium at 2 mg/ml in order to sequester DNA-interacting compounds produced by actinomycetes. The presence of these DNA-interacting compounds can cause a high screening background when DNA damaging agents are not targets of drug screening. In addition, one can add D-biotin at 0.1 μg/mL to overcome the negative effect of biotin-sequestering compounds released by actinomycetes, especially if using screening strains auxotrophic for biotin. The procedure of adding DNA into the culturing medium should be omitted when DNA damaging agents are targeted in drug screening. In an embodiment using CM400 as the screening strain, the matrix for the bioassay can be generated with Muller-Hinton agar at a concentration of 1×10⁶CFU/ml E. coli CM400. The culture media contain counter-selective agents, for example, Ampicillin 50 μg/mL, Tetracycline 10 μg/mL, and Daptomycin 20 μg/mL, to prevent the contamination by non-actinomycete bacteria that may grow in, on or around the macrodroplets during incubation. Approximately 100 mL of this matrix can be poured onto the colonized gel beads per 25×25 cm bioassay dish. The volume of the matrix can be variable and should depend on the size of the bioassay dish. During the overnight incubation period, the CM400 will grow in the matrix, except in areas where macrodroplet beads release metabolites, which can inhibit the growth of CM400, resulting in zones of inhibition around these beads.

When a skilled person chooses to use a screening strain containing reporter fusion gene(s) in the screening procedure, it is preferable to apply methods for detecting the reporter gene(s) expression in order to determine positive MDs. For example, if one chooses to use a screening strain having lacZ as the reporter gene, one can add substrates of β-galactosidase to the medium and easily detect positive MDs since test cells surrounding positive MDs will turn blue. Methods for detecting commonly used reporters, such as GFP, RFP, luciferase, are all well known by a skilled person in the art.

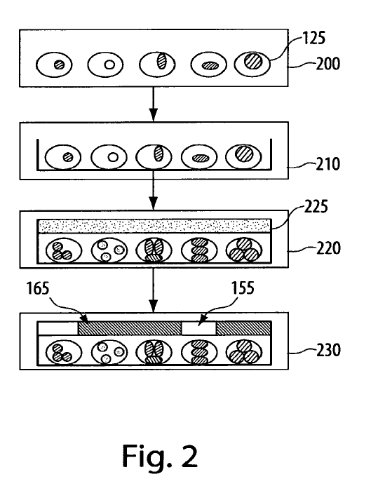

When one skilled person chooses to use cell lines as screening strains, a two-layer agar diffusion bioassay can be applied. Procedures of the above assay is schematically illustrated in FIG. 2. MDs (FIG. 2, item 125) should first grow in bioassay plates (FIG. 2, item 200). After culturing for a certain period of time, they then can be embedded in 0.6% agar (FIG. 2, item 210). The culturing time and condition should depend on the type of microorganisms targeted. For example, the culturing time for actinomycetes is preferred to be 7 days at 28° C. The plate can then be overlayed by 0.8% low melting agarose in EMEM medium (item 225 in FIG. 2), in which the screening cells should be inoculated. After incubating for 48 hours, 3-(4,5-dimethylthiazol-2-yl)-2,5-diphenyltetrazolium bromide (MTT) can be added to the agarose surface and cytotoxicity can be visualized so that viable cells should be stained purple (FIG. 2, item 165) while toxicity should be observed as zones of clearing (FIG. 2, item 155). See FIG. 2 item 230. Also see section 5.3.5 for more details.

5.2.3.4. Recovery and Identification of Microorganisms Producing Useful Natural Compounds

Beads situated in inhibition zones, or inside the zones where reporter genes are expressed, can be recovered from the matrix. First, these beads are washed and incubated in a solution containing an antibiotic, such as aztreonam, to which only the screening strain is sensitive, thereby reducing carry over of the screening strain to the next stage. Surface-sterilized beads are then allocated into multi-well plates and dissolved in a solution of sodium citrate. The liquid phase is removed by filtration under vacuum and the biomass is incubated with a nutrient solution, containing further antibiotics to eliminate any remaining presence of the screening strain. The biomass is replica plated to a multi-well microfermentation plate containing nutrient medium. Microorganisms present in the macrodroplets are then allowed to grow in the microfermentation plates after which a second copy is made and stored frozen in cryoprotectant. Single purified microorganisms are further characterized or dereplicated using a variety of techniques, including phenotypic or metabolic characterization of strains, ribotyping, chemical analysis and 16S rDNA gene sequencing.

One set of the microfermentation plates can then be extracted for testing against a panel of anti-microbial strains to confirm the recovered microbial strains can produce antimicrobial agents. Microorganisms producing anti-microbial agents can be accessed from the copy plates, their purity confirmed, and stored individually at −135° C. Characterization of actinomycetes by 16S rDNA analysis provides phylogenetic information about the actinomycetes isolated as hits from soils and the relationship between them at a sub-species level. The diversity of the hits obtained can be compared for soils encapsulated under different conditions and from different soil sources. Organisms that appear to be replicates on the basis of 16S rDNA sequence can, if desired, be further analyzed for strain specific differences.

The active compounds of interest can be purified from microorganism cultures and subjected to physical and chemical investigations and biological assays, well known to persons skilled in these arts, to determine their chemical structures, physico-chemical properties and biological activities. See FIG. 1, item 160.

5.2.3.5. A Method for Recovering Actinomytes from Macrodroplets

An embodiment of recovering actinomycetes from the positive macrodroplets is discussed in detail below. Typically, beads were washed up to 3 times in 10% glycerol containing 20 μg/mL aztreonam which were then drained and transferred individually to v-bottomed 96-well plates containing 150 μL 0.25M sodium citrate buffer with 20 μg/mL aztreonam. The plates were shaken for 30 minutes during which time the alginate beads were dissolved to release biomass. Aliquots of released biomass were transferred to the surface of 25 mm 0.2 μm cellulose nitrate filters sitting on 6-well plates containing oatmeal and yeast extract based medium. The plates were then incubated at 28° C. for a period of 3-4 days, after which time the filters were removed, leaving the actinomycete mycelia, which had penetrated the membrane, and colonised the medium below. Actinomycete colonies were then incubated for up to a further 7 days at 28° C. before subculturing onto nutrient medium for further characterization.

Actinomycetes growing on the oatmeal and yeast extract based medium were further purified if necessary to obtain single cultures. Phenotypical dereplication was conducted at this stage to reduce the number of actinomycete strains that were subject to further characterization. Replicates of the identical actinomycete strains were detected by their physical phenotypes, such as color and morphology, and their chemical phenoytypes as determined by extraction and analysis by HPLC and HPLC-MS of their metabolites. Only one or two of these identical clones was selected for further characterization. Once axenic cultures were obtained, a further screen was performed to check whether the actinomycete strain purified was truly able to produce natural compounds that inhibit the growth of the screening strain. This assay was carried out by growing the actinomycete strain selected for a defined period of time (ranging from 7-14 days, preferable 7 days, on nutrient agar medium and then removing a plug of the biomass and agar which was subsequently placed on a lawn of E. coli CM400 as follows. The innoculum of CM400 was prepared by growing 5 mL of sterile MHB in a 50 mL flask until an OD₆₀₀ₙₘ 0.1 is reached (equivalent of 2×10⁸ cfu/mL). The culture was double diluted in sterile MHB to give a working stock of 1×10⁸ cfu/mL.

A sterile cotton swab was then dipped into the adjusted well-mixed suspension and streaked over two-thirds of surface of the sterile 245×245 mm ‘Nunc’ dish plate containing Mueller-Hinton agar with appropriate additives (e.g. +0.2% DNA, +20 mg/L biotin). This procedure was repeated by streaking the swab three more times, rotating the plate by 90° each time to ensure an even distribution of inoculum. The plates were air-dried for 5 minutes. Finally, an agar plug was cut from the isolate plate and placed on the assay plate. The plug should be approximately 5×5 mm in size and taken from an area of the culture that shows both mature growth and young growth so that a good cross-section of the culture age is sampled.

Actinomycete strains with confirmed capacity of useful compounds production were fermented in a larger volume suitable for species determination and compound purification. The genus and species of the selected actinomycete strains were determined by 16S rDNA gene sequencing. Compounds of interests were purified from the culture by standard methods known in the art. Purified compounds were further characterized by various physical, chemical and biological assays to determine their chemical structures, physical properties and bioactivities

### 5.3. Specific Examplification of the Invention

5.3.1. Specific Examples of Screening Strains and the Construction Processes Thereof:

5.3.1.a. Escherichia coli Screening Strain, DR1234: Multiple Drug Resistances.

The Escherichia coli screening strain, DR1234, containing both multiple drug-resistant genes (see Table 5) and auxotrophic mutations as well (see Table 6), has been constructed by genetically modifying the parent strain, MG1655 (E. coli Genetic Strock Center #7740) whose genome has been sequenced in its entirety, Blattner et al., 1997 Science, 277(5331):1453-74), according to the following procedures.

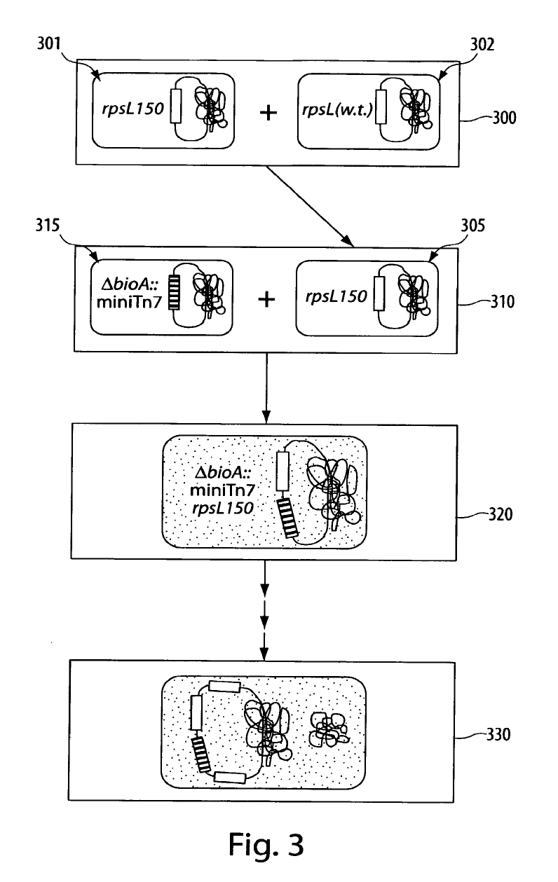

The drug resistant genes to be introduced were obtained from both academic and commercial sources (listed in Table 5.) The drug resistance genes associated with Tn7 were contiguous and introduced as a modified cassette in which the cryptic sat promoter region was replaced by a synthetic promoter P_{trc}. Amnan et al. 1988, Gene 30; 69(2):301-15). The modified cassette is termed “miniTn7+”. See FIG. 3, item 315.

TABLE 5
Drug-resistant Markers Contained in Screening Strain DR1234.

| Drug resistance | Gene | Source | Supplier | GenBank # |
|---|---|---|---|---|
| Aminoglycosides** | aph(2″)-Ib | E. faecium | Joseph Chow* | AF207840 |
| Apramycin | aac(3′)-IV | pStreptoBACV | Cubist | AJ414670 |
| β-lactams | TEM-1 bla | PQEΔNde2 | Cubist^{#} | X54604 |
| Chloramphenicol | cat | pKD3 | E. coli Genetic Stock Center | AY048742 |
| | | | (#7631) | |
| Nalidixic acid | nalA37 | UB1005 | E. coli Genetic Stock Center | D90854 |
| | | | (#7138) | |
| Neomycin | neo | pSU2007 | F. de la Cruz†† | V00618 |
| Spectinomycin | aadA1 | Tn7† | DSMZ 3872 (colE1::Tn7 | AP002527 |
| | | | plasmid) | |
| Streptomycin | aadA1 | Tn7† | DSMZ 3872 (colE1::Tn7 | AP002527 |
| | | | plasmid) | |
| Streptomycin | rpsL150 | MC4100 | E. coli Genetic Stock Center | AF312716 |
| | | | (#6152) | |
| Streptothricin | sat | Tn7 | DSMZ 3872 (colE1::Tn7 | AP002527 |
| | | | plasmid) | |
| Trimethoprim | dhfrI | Tn7† | DSMZ 3872 (colE1::Tn7 | AP002527 |
| | | | plasmid) | |
| Tetracycline | metE::tetA | CAG18491 | E. coli Genetic Stock Center | J01830 |
| | | | (#7464) | |
| Trimethoprim | dfrB2 | pSU2007 | F. de la Cruz†† | X01702 |

†The drug resistance genes associated with Tn7 are contiguous and introduced as a modified cassette in which the cryptic sat promoter region is replaced by a synthetic promoter (Ptrc; Amman et al., 1988, Gene 30; 69(2): 301-15). The modified cassette is termed “miniTn7+”.
††Martinez and de la Cruz, 1988, Mol Gen Genet., 211(2): 320-5. Plasmid pSU2007 is a sulfonamide-sensitive derivative of R388 that contains the Tn5 neo gene (F. de la Cruz, personal communication).
^{#}Modified from pQE60 (Qiagen, Inc.): Created an Nde1 resitriction site at position 114 by fusion with blunt ended Nco1 site “ccatatg” and eliminated the Nde1 site at position 1369 by insertion of nucleotide in restriction site “cattatg”.
*Kao et. al., (2000) Antimicrob. Agents Chemother., 44(10): 2876-2879.
**include gentamycin, tobramycin, dibekacin, netilmycin, kanamycin-A, arbekacin

Alleles containing some of the listed drug resistant genes (underlined in Table 5) already exist at certain chromosomal loci in other E. coli strains (nalA37, rpsL150, metE::tetA). These alleles were introduced into MG1655 in an iterative manner using standard bacteriophage P1 transduction method, which was described in detail in “A Short Course in Bacterial Genetics”, 1992, ed. Miller, Cold Spring Harbor Press, pp. 268-274. FIG. 3 provides a schematic illustration of how drug-resistant genes have been tranduced from donor E. coli strains to the recipient strain, MG1655. In FIG. 3, item 300, the P1 lysate of the donor strain (the strain containing the drug resistance gene to be transferred, e.g. rspL150, FIG. 3, item 301) was made by growing one colony of the donor strain in 1 ml LB broth, at 37° C. overnight with agitation. This culture was then diluted at the ratio of 1:100 into 1 ml fresh LB broth with 4% glucose and 0.005M CaCl₂. The diluted culture was incubated at 37° C. in a static water bath for 20 minutes, then 20 ul of a stock P1 lysate (grown on MG1655) was added. The infected culture was grown at 37° C. with aeration until lysis was evident. 50 ul CHCl₃ was added to the lysate which was then vortexed for 30 s. Cell debris was removed by centrifuging the lysed culture in an Eppendorf microcentrifuge, room temperature at 5,000×g for 5 minutes.

Phages in this lysate were used as the vehicle to transfer the donor drug resistance genes into the recipient strain (MG1655 in this case, FIG. 3, item 302). Briefly, the recipient strain was grown from a single colony overnight in 5 ml LB broth medium at 37° C. The culture was then centrifuged at 2300×g at room temperature to spin down the cells. The cell pellet was resuspended in MC buffer (0.1M MgCl₂, 0.005M CaCl₂) and incubated with agitation at 37° C. for 20 min. Twenty microliter (20 ul) of the donor lysate was combined with 100 ul of the resuspended culture and incubated at 37° C. for 20 minutes, after which 4 ml LB broth plus 200 ul 1M Na-citrate was added. This mixture was incubated for a further 1 h at 37° C. with agitation. Cells were then harvested by centrifuging at 2300×g at room temperature. The cell pellet was resuspended in 150 ul 1M Na-citrate and spread on LB agar plates that were selective for the donor drug resistance. After overnight incubation at 37° C., drug-resistant colonies were purified by streaking on selective LB agar plates containing 0.005M Na-citrate and incubation overnight at 37° C. Drug resistant colonies were assessed for presence of the new allele by polymerase chain reaction (PCR) diagnostic assay. This was performed by a standard PCR assay as described in Roche's High Fidelity PCR System package, using synthetic oligonucleotide primers designed to anneal to and amplify the region of the chromosome where the new allele was located. PCR using such primers would give either a different sized fragment, or a fragment with a different sequence, as compared to the wild-type parental strain. Once confirmed, the new drug resistant strain could be used as a recipient for additional drug resistance genes.

Part of the drug-resistant genes, for instance, Tn7-associated genes (italic in Table 5, also see FIG. 3, item 310, e.g. aac(3)-IV) were further introduced by standard DNA homologous recombination method described by Datsenko and Wanner, 2000, Proc. Nat. Acad. Sci. 97(12):6640-6645. See U.S. Pat. No. 6,355,412 and U.S. Pat. No. 6,509,156. Donor E. coli. strain used in the P1 phage-mediated gene transduction as described above also contained some of these genes (both underlined and italic in Table 5); however, they were in mutated forms. Therefore, a functional copy was introduced by homologous recombination as described hereafter. The drug-resistant gene of interest was synthesized in vitro using PCR with oligonucleotide primers that contained homologous sequences to the target genes and an additional ˜50 by homologous to a particular chromosomal site of the recipient strain. For instance, to insert the Tn7-associated resistance gene cassette dhfrI-sat-aadA into the bioA gene locus of the recipient strain, the primers listed in Table 6 were used. The capitalized portions of these primers hybridized to Tn7 template DNA upstream and downstream of the resistance gene cassette and allowed PCR amplification of the target fragment (Roche (Mannheim, Germany) Long Template PCR System). The ˜50 by additional sequence of these oligonucleotides (low case) afforded regions of homology with the chromosomal target site of the recipient, in this instance, the bioA gene locus (Table 6). The PCR product of the above reaction was introduced into a specialized strain, BW25113 (obtained from the E. coli Genetic Stock Center, strain # 7739) by standard electroporation following the manufacturer's instruction of BioRad (Hercules, Calif.) Gene Pulser, model #1652076, for standard electroporation of E. coli. Double-crossover homologous recombination, mediated by the bacteriophage lambda Red recombinase present in the strains pKD46 [#7630], pKD78 [#7989] or pKD119 [#7990], occurred between the “bioA” locus of the recipient strain and the PCR products via their homologous regions designed in the PCR primers as described above, resulting in the insertion of the drug resistance cassette into the bioA locus of the chromosome (FIG. 3, item 315). The advantage of this strategy was that it not only introduced drug-resistant genes, but also deleted majority of the bioA gene, thus rendering the recipient strain both drug-resistant and auxotrophic (rely on biotin). Miller, et al., p. 437. Once verified by drug resistance characterization and diagnostic PCR assay (see above), the drug resistance-encoding locus from the BW25113/pKD46 strain was introduced into the same locus of the recipient screening strain by standard P1 transduction as described above, also see FIG. 3, item 310.

The BW25113 strain containing drug-resistant genes inserted into the bioA locus by homologous recombination as described above were found to confer resistance to trimethoprim (at least 10 ug/ml; by virtue of the dhfrI gene) but not to streptothricin or spectinomycin (at 5 ug/ml and 100 ug/ml, respectively). It has been reported that the sat gene in the Tn7 context was cryptic due to inefficient expression. See Sundstrom et al. 1991, “Site-specific insertion of three structural gene cassettes in transposon Tn7,” J. Bacteriol. 173(9):3025-3028; Tietze & Brevet, 1991, “The trimethoprim resistance transposon Tn7 contains a cryptic streptothricin resistance gene,” Plasmid, 25:217-220. To increase the gene expression level, the T7 promoter was used to replace the weak promoter of sat gene. The T7 promoter region (P_{trc}) was amplified using oligonucleotide primers which contained regions homologous to the upstream of sat gene (in lower case), such that the recombination reaction according to Datsenko and Wanner scheme resulted in replacement of the native sat promoter region with the synthetic trc promoter. See U.S. Pat. No. 6,355,412 and U.S. Pat. No. 6,509,156. The resulting strains having the T7 promoter substitute were resistant to both streptothricin and spectinomycin. The whole mini Tn7 gene cassettes driven by the T7 promoter was introduced into the intermediate screen strain (MG1655(rpsL150 in FIG. 3, item 305) by P1 phage mediated gene transduction. See FIG. 3, item 310.

TABLE 6
Disruption/replacement primers to generate resistance
cassettes used in homologous recombination by the
method described by Datsenko and Wanner.

| | | Drug-resistant | | |
|---|---|---|---|---|
| | | gene(s)/ | | |
| Targeting | Accession | promotor | Primer | |
| locus | No. | inserted | name | Primer sequence † |
| bioA | J04423 | Tmp * | Tn7upbio | atgacaacggacgatcttgcctttga |
| | | | | ccaacgccatatctggcacccatacG |
| | | | | AGATGCTTGTTTACCGGTAG |
| | | | Tn7dnbio | atgtttcatcctgtaccgcgcggtta |
| | | | | accgctgcggtcagacgctgcaacCC |
| | | | | AGCTCTCTAACGCTTGAG |
| csgA | X90754 | aac(3')-IV | aac- | GAAACTTTTAAAAGTAGCAGCAATTG |
| | | | st_csg5 | CAGCAATCGTATTCTCCGGTAGCGCA |
| | | | | GGTCGCTCACGGTAACTG |
| | | | aac- | CAGTGGAACGGCAAAAATTCTGAAAT |
| | | | end_csg3 | GACGGTTAAACAGTTCGGTGGTGGTC |
| | | | | TCCGCTCATGAGCTCAGC |
| ilvG | M10313 | cat | ilvG-cat5 | CCGAAGTTGAGCAAGCGCGCCAGATG |
| | | | | CTGGCAAAAGCGCAAAAACCGATGGG |
| | | | | CGCGCCTACCTGTGACGG |
| | | | ilvG-cat3 | CGTAACGCCAGGAATGTTCATCACGC |
| | | | | AGCTGCGCGCAGTGTTGCTGCCAGTC |
| | | | | ATCGCAGTACTGTTGTAT |
| Sat | AP002527 | “P_{Trc}” ** | satproup | atagttaccaaatctggcaaaagggt |
| promoter | | | | taacaagtggcagcaacggattcgCT |
| | | | | GGCAAATATTCTGAAATGAG |
| | | | satprodn | ggttctcagcatccaatgtttccgcc |
| | | | | acctgctcagggatcaccgaaatctt |
| | | | | catCTATACTTCCTCCTGGGTACC |

† Sequences in lower case are homologous to the corresponding target locus (for homologous recombination) and sequences in uppercase are designed for PCR amplification of the corresponding resistance marker(s).
* TmpR gene is part of the Tn7 resistance cassette.
** Ptrc is the T7 promoter, which replaces sat promoter and drives the expression of sat and aad genes in the resultant screening strain.

As described above, the drug-resistant cassettes were inserted into some gene locus responsible for the biosynthesis pathway of certain essential nutrients, resulting in the recipient strain being auxotrophic. In addition to the bioA locus, csgA gene responsible for curli synthesis and ilvG gene responsible for the synthesis of Ile and Val were also the targeting loci. The aac(3′)-IV cassette was used to delete the csgA gene in the curli locus and cat gene to disrupt ilvG in the ilv locus (Table. 6). Also see FIG. 3, items 320 and 330.

Two plasmids containing the rest of the resistance markers listed in Table 5 were isolated from strain Ec 744Tmp using standard DNA isolation techniques, see Molecular Cloning, 1989, Sambrook et al., Cold Spring Harbor Press, pp. 1.25-27, and then transformed into the appropriate MG1655 derivatives by standard electroporation. See FIG. 3, item 330.

The screening strain, DR1234, was established after all desired drug-resistant genes listed in Table 5 had been delivered into the parent E. coli strain, MG1655, by methods described above. Its level of drug resistance was analyzed by the standard minimum inhibitory concentration (MIC) assay in MHB liquid medium (Chantot, et al., 1986, “Antibacterial activity of roxithromycin: a laboratory evaluation,” J. Antibiot, 39(5):660-8) and the result was summarized in Table 7.

TABLE 7
Minimal inhibitory concentrations (MIC₉₀) of antibiotics
against E. coli MG1655 and DR1234 in MHB medium.
DR1234 was resistant to drugs whose resistant genes had
been incorporated into it, but not to those whose corresponding
resistant genes were not present in this screening strain.

| Antibiotic | MG1655 | DR1234 |
|---|---|---|
| Actinomycin C | 50 | 100 |
| Actinomycin D | 200 | 100 |
| Ampicillin | 1.6-3.2 | >400 |
| Apramycin | 3.2-6.3 | 400 |
| Aztreonam | 0.1 | ≦0.05 |
| Bleomycin | ≦0.2-0.4 | 0.8 |
| Chloramphenicol | 3.2 | 100 |
| Coumermycin A1 | 12.5 | 3.2 |
| Daunorubicin | 200 | >400 |
| Erythromycin | 25 | 100 |
| Gentamicin | 0.8 | 400 |
| Kanamycin | 0.8-1.6 | >400 |
| Mitomycin C | 0.8 | 3.2 |
| Nalidixic Acid | 6.3 | 400 |
| Neomycin | 0.8 | 100 |
| Netilmycin | 1.6 | 200 |
| Spectinomycin | 6.3-12.5 | >400 |
| Streptomycin | 3.2 | >400 |
| Streptonigrin | 3.2 | 3.2 |
| Streptothricin | 0.4-1.6 | 400 |
| Tetracycline | 0.4-3.2 | 100 |
| Tobramycin | 0.4 | 50 |
| Trimethoprim | ≦0.4 | >400 |

5.3.1.b. E. coli Screening Strain CM166: Multiple Drug Resistances and Auxotrophies.

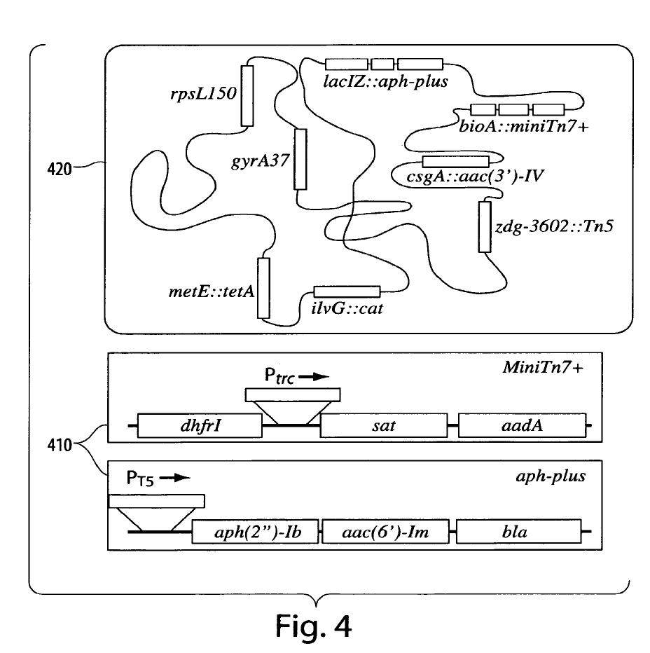

The screening strain, CM166, is a plasmid-free multiple drug resistant Escherichia coli strain that is constructed according to the following procedures. The drug resistance genes incorporated into CM166 were obtained from both academic and commercial sources as shown in Table 8. Item 410 in FIG. 4 shows the expression cassettes of sat-aadA driven by P_{trc} and aph(2″)-'Ib-aac(6′)-Im-bla driven by P_{T5} promoter. Item 420 further details the multiple drug resistance genes which are incorporated into the screening strain CM166. The detailed procedures of constructing CM166 is described below.

TABLE 8
Relevant resistance markers

| Drug resistance | Gene | Source | Supplier | GenBank |
|---|---|---|---|---|
| Aminoglycosides** | aph(2″)-Ib | E. faecium | Joseph Chow* | AF207840 |
| | aac(6′)-Im | | | AF337947 |
| Apramycin | aac(3′)-IV | pStreptoBACV | Cubist | AJ414670 |
| β-lactams | TEM-1 bla | PQEΔNde2 | Cubist*** | X54604 |
| Chloramphenicol | cat | pKD3 | E. coli Genetic Stock Center | AY048742 |
| | | | (#7631) | |
| Nalidixic acid | nalA37 | UB1005 | E. coli Genetic Stock Center | D90854 |
| | | | (#7138) | |
| Neomycin | neo | Tn5 | E. coli Genetic Stock Center, | V00618 |
| | | | #7382 | |
| Spectinomycin | aadA1 | Tn7† | DSMZ 3872 (colE1::Tn7 | AP002527 |
| | | | plasmid) | |
| Streptomycin | aadA1 | Tn7† | DSMZ 3872 (colE1::Tn7 | AP002527 |
| | | | plasmid) | |
| Streptomycin | rpsL150 | MC4100 | E. coli Genetic Stock Center | AF312716 |
| | | | (#6152) | |
| Streptothricin | sat | Tn7 | DSMZ 3872 (colE1::Tn7 | AP002527 |
| | | | plasmid) | |
| Trimethoprim | dhfrI | Tn7† | DSMZ 3872 (colE1::Tn7 | AP002527 |
| | | | plasmid) | |
| Tetracycline | metE::tetA | CAG18491 | E. coli Genetic Stock Center | J01830 |
| | | | (#7464) | |
| Bleomycin | ble | Tn5 | E. coli Genetic Stock Center | X01702 |
| | | | (#7382) | |

*Kao et. al., 2000, Antimicrob. Agents Chemother., 44(10): 2876-2879.
**including gentamycin, tobramycin, dibekacin, netilmycin, kanamycin-A, arbekacin
***Modified from pQE60 (Qiagen, Inc.): Created an Nde1 resitriction site at position 114 by fusion with blunt ended Nco1 site “ccatatg” and eliminated the Nde1 site at position 1369 by insertion of nucleotide in restriction site “cattatg”.

E. coli strain MG1655 (E. coli Genetic Stock Center # 7740), whose genome has been sequenced in its entirety (Blattner et al., (1997) Science. September 5; 277(5331):1453-74), was chosen as the parent strain for the CM166. The following two gene transfer strategies were taken to introduce the drug resistance genes into this strain.

Some of these drug resistance genes (other than the ones derived from Tn7 sources) are alleles already present at chromosomal loci in other E. coli strains (nalA37, rpsL150, metE::tetA, Tn5 (neo, ble)). These alleles are introduced into MG1655 in an iterative manner using standard bacteriophage P1 transduction using a P1 phage lysate made on a donor strain containing the drug resistance gene. See A Short Course in Bacterial Genetics 1992, ed. Miller, Cold Spring Harbor Press, pp. 268-274 and section 5.3.1 for experimental details. Also see FIG. 3. The drug-resistance phenotype of the recipient strains was confirmed by PCR amplification assay and drug selection assay (the viability of the strain in medium containing the corresponding antibiotics). Once confirmed, the resulting strain (the intermediate strain) was used as the recipient strain for the further delivery of additional drug resistance genes and other genetic traits.

Resistance genes that do not reside on the chromosome to start with (for instance, Tn7-associated genes, aph(2″)-Ib, aac(6′)-Im, TEM-1 bla and aac(3′)-IV) were introduced into the screening strain, or precursor strains, using the method described by Datsenko amd Wanner, 2000. See U.S. Pat. No. 6,355,412; U.S. Pat. No. 6,509,156. The resistance gene(s) of interest were synthesized in vitro by PCR amplification using oligonucleotide primers that contain both sequences for PCR amplification as well as approximately 50 by homologous sequences to a chromosomal target site.

For instance, the resistance cassettes encoding bla, aph(2″)-Ib and aac(6′)-Im were combined into a new composite resistance cassette and the expression was driven by a T5 promoter. Specifically, the aph(2″)-Ib and aac(6′)-Im genes were amplified by PCR as a single linked unit from E. faecium SF11770 and ligated into the Nde1/BamH1 sites of the pQEΔNde2 in frame with the T5 promoter. The sequences of the oligonucleotide primers were: “aph2-Nde1” 5′-cggcgcatatgGTTAACTTGGACGCTGAG-3′ and “aph2-BamH1” 5′-cggegggatccTTACACTCTCCATTCCATCAG-3′. The expression of aph(2″)-Ib and aae(6′)-Im in the resulting plasmid, pCM100, was under the control of the T5 promoter. A third resistance gene, bla, was amplified with the oligonucleotide primers “bla+-BamH1” 5′-cggcgggatccGCTCATGAGACAATAACCCTG-3′ and “bla-Nhe1” 5′-cggcggctagcTTACCAATGCTTAATCAGTG-3′ using pQEΔNde2 as the template. The amplified fragment contained an additional 55 nucleotides upstream of the bla transcription start site providing a spacer region between the end of aac(6′)-Im and the start of bla in the final multiple-gene-cassette. The bla fragment was ligated into the BamH1/Nhe1 sites of pCM100, generating the construct, pCM101, which contained the multiple antibiotic resistant gene cassette (P_{T5} aph(2″)-Ib-aae(6′)-Im-bla=aph-plus; see FIG. 4). The entire cassette including the T5 promoter was amplified using pCM101 as the template and the fragment was then used to replace chromosomal lacIZ by the method described by Datsenko and Wanner, 2000. See U.S. Pat. No. 6,355,412; U.S. Pat. No. 6,509,156. The following oligonucleotide primers were used for the lacIZ locus disruption: “T5-aph2-1” 5′-ggtggccggaaggcgaageggcatgcatttacgttgacaccatcgaatggAAATCATAAAAAATTTATTTG-3′ and “Wbla-1” 5′-gtacataatggatttccttacgcgaaatacgggcagacatggcctgcccgg TTACCAAT GCTTAATCAGTG-3′. The sequences in uppercase hybridized to the multiple drug resistant cassette while the sequences in lower case were homologous to the lacIZ locus and mediated the site-specific homologous recombination. The distribution of the resistance alleles and cassettes is shown in FIG. 4, item 420. The disruption of bioA and metE loci resulted in biotin and methionine auxotrophies.

The level of drug resistance of CM166 was estimated by MIC assay carried out in MHB liquid medium and the results were summarized in Table 9. CM166 is resistant to antibacterial agents whose resistant genes have been incorporated into the strain, but not to other antibacterial drugs whose resistant genes are not incorporated.

TABLE 9
Minimal inhibitory concentrations (MIC₉₀) of antibiotics
against E. coli MG1655 and CM166 in MHB medium.

| Antibiotic | MG1655 | CM166 |
|---|---|---|
| Actinomycin C | 50 | 50 |
| Actinomycin D | 200 | 50 |
| Ampicillin | 1.6-3.2 | >400 |
| Apramycin | 3.2-6.3 | 200 |
| Aztreonam | 0.1 | ≦0.05 |
| Bleomycin | ≦0.2-0.4 | 400 |
| Chloramphenicol | 3.2 | 100 |
| Coumermycin A1 | 12.5 | 6.3 |
| Daunorubicin | 200 | >400 |
| Erythromycin | 25 | 100 |
| Gentamicin | 0.8 | >400 |
| Kanamycin | 0.8-1.6 | >400 |
| Mitomycin C | 0.8 | 3.2 |
| Nalidixic Acid | 6.3 | 800 |
| Neomycin | 0.8 | 25 |
| Netilmycin | 1.6 | 400 |
| Spectinomycin | 6.3-12.5 | >400 |
| Streptomycin | 3.2 | >400 |
| Streptonigrin | 3.2 | 1.6 |
| Streptothricin | 0.4-1.6 | 400 |
| Tetracycline | 0.4-3.2 | 100 |
| Tobramycin | 0.4 | >400 |
| Trimethoprim | ≦0.4 | >400 |

5.3.1.c. E. coli Screening Strain DR1212: Supersensitive to DNA Damaging Agents

Compounds that can cause DNA damage are potential anti-tumor drug candidates because tumor cells, as fast growing cells, are much more sensitive to such drugs. These compounds can be obtained by screening bacterial strains, which bear non-functional mutation in the DNA repair system; therefore are hypersensitive to DNA damage. This example teaches the construction of screening strain DR1212, which contains multiple drug resistant genes similar to DR1234 (see section 5.3.1.a), and additionally, the as recA mutation, which results in hypersensitivity to DNA damaging agents.

E. coli strain MG1655 (E. coli Genetic Stock Center # 7740), whose genome has been sequenced in its entirety (Blattner et al., (1997) Science, 277(5331):1453-74), was chosen as the parent strain for DR1212. Multi-drug resistant genes were incorporated into the parental strain by methods described in section 5.3.1.a. Briefly, these drug resistant genes were delivered by P1 phage mediated gene transduction, homologous recombination, and plasmid transformation.

An additional recA mutation was further introduced after drug-resistance genes had been incorporated. The mutated recA locus, in which a cat gene was inserted (see Table 10), was derived from an E. coli strain described in Wanner & Boline, 1990, “Mapping and molecular cloning of the phn (psiD) locus for phosphonate utilization in Escherichia coli,” J. Bacteriol., 172:1186-1196. The mutated recA locus was introduced into the intermediate strain (containing all drug-resistant genes) by standard P1 phage mediated gene transduction, establishing the target screening strain, DR1212.

TABLE 10
Additional relevant resistance markers

| Drug resistance | gene | source | supplier | GenBank # |
|---|---|---|---|---|
| Chloramphenicol | cat | pKD3 | E. coli Genetic | AY048742 |
| | | | Stock Center (#7631) | |

The level of drug-resistance of screening strain DR1212 was analyzed by standard MIC assay in MHB liquid medium and the result was summarized in Table 11. Similar to screening strains, DR1234 and CM166, DR1212 is also resistant to drugs whose resistant genes have been introduced.

TABLE 11
Minimal inhibitory concentrations (MIC₉₀) of antibiotics
against E. coli MG1655 and DR1212 in MHB medium.

| Antibiotic | MG1655 | DR1212 |
|---|---|---|
| Actinomycin C | 50 | 25.0 |
| Actinomycin D | 200 | 50.0 |
| Ampicillin | 1.6-3.2 | >400 |
| Apramycin | 3.2-6.3 | 1.6 |
| Aztreonam | 0.1 | ≦0.05 |
| Bleomycin | ≦0.2-0.4 | ≦0.4 |
| Chloramphenicol | 3.2 | 400 |
| Coumermycin A1 | 12.5 | 3.2 |
| Daunorubicin | 200 | 12.5 |
| Erythromycin | 25 | 50 |
| Gentamicin | 0.8 | 200 |
| Kanamycin | 0.8-1.6 | 400 |
| Mitomycin C | 0.8 | <0.4 |
| Nalidixic Acid | 6.3 | 50 |
| Neomycin | 0.8 | 25 |
| Netilmycin | 1.6 | 400 |
| Spectinomycin | 6.3-12.5 | >400 |
| Streptomycin | 3.2 | >400 |
| Streptonigrin | 3.2 | ≦0.2 |
| Streptothricin | 0.4-1.6 | 200 |
| Tetracycline | 0.4-3.2 | 100 |
| Tobramycin | 0.4 | 25 |
| Trimethoprim | ≦0.4 | >400 |

5.3.1.d. E. coli. Screening Strains CM191 and CM242: lacZ Reporter Genes Driven by SOS-Response Promoter sulA and recN, Respectively

In addition to the construction of screening strains containing multiple drug resistant genes and auxotrophic mutations, we have shown in this example the construction of screening strains having a reporter gene fused with a promoter sequence responsive to specific physiological conditions, such as DNA damage, translation inhibition, etc., for the purpose of selecting for/against compound classes with a specific mechanism of action. In particular, we have constructed screening strains containing bacterial SOS-response promoters of sulA and recN fused with LacZ reporter gene, respectively, to detect DNA damaging compounds from natural sources. Quillardet and Hofnung, (1993), Mutation Research, 297:235; Khil and Camerini-Otero, (2002), Mol Microbiol 44:89.

P_{sulA}′-'lacZ reporter fusion gene was constructed according to the following procedures. The multiple drug resistant cassette (P_{T5} aph(2″)-Ib-aac(6′)-Im-bla) of strain CM166, as described in section 5.3.1.b, was amplified by PCR and the fragment was then inserted 62 nucleotides upstream of the E. coli MG1655 sulA promoter (AE000198 bases 2558-2625) by homologous recombination described by Datsenko and Wanner, 2000. See U.S. Pat. No. 6,355,412 and U.S. Pat. No. 6,509,156. The following oligonucleotide primers, sulA-W4″ and “sulA-W5”, see Table 12, were used for PCR amplification of the corresponding cassette and the subsequent recombination. The regions of these primers in uppercase hybridizes to the (P_{T5} aph(2″)-Ib-aac(6′)-Im-bla) cassette while the regions in lower case were homologous to the upstream sequence of the sulA promoter and mediated homologous recombination at the target locus. The drug-resistant genes were in opposite orientation to sulA promoter, and the recombination also replaced b0959 (AE000198 bases 2776-3405). Homologous recombination resulted in the intermediate strain, CM177, which was then used as the template for PCR amplification of a fragment containing both the drug-resistant genes cassette (P_{T5} aph(2″)-Ib-aac(6′)-Im-bla) and the sulA promoter. The PCR fragment was generated using the following oligonucleotide primers, lacZ/sulAW1 and lacZ/sulAW2 in Table 12, which was designed to mediate the insertion of the whole PCR product into the lacIZ locus. The regions of these primers indicated in uppercase hybridized to the drug-resistant genes cassette while the regions indicated in lower case bore homology to the lacIZ locus. The recombination resulted in the fusion of P_{sulA} to the lacZ reporter gene. In the resulting strain, CM191, both lacI and the region between lacI and lacZ were replaced by the P_{sulA}-(P_{T5} aph(2″)-Ib-aac(6′)-Im-bla) cassette and the expression of lacZ was completely under the control of the sulA promoter.

TABLE 12
Disruption/replacement primers used to
generate the drug-resistant cassettes with
or without targeted promoter regions:

| Primer name | sequence |
|---|---|
| sulA-W4 | gagcctcgcaaattttgtcgttggtgacgggaa |
| | aacataaattaatcttgAAATCATAAAAAATTT |
| | ATTTG |
| sulA-W5 | gttggattattgattgcattactgcctgagatt |
| | ggtcgtctgcaacgccagTTACCAATGCTTAAT |
| | CAGTG |
| lacZ/sulAW1 | cagtcacgacgttgtaaaacgacggccagtgaa |
| | tccgtaatcatggtcatAATCAATCCAGCCCCT |
| | GTGAG |
| lacZ/sulAW2 | gcggtatggcatgatagcgcccggaagagagtc |
| | aattcagggtggtgaatTTACCAATGCTTAATC |
| | AGTG |
| pBR-GR-recN1 | ttagaataatttttttgaccagccgagcttggt |
| | gcttaatgtgttgaaatTTCTTAGACGTCAGGT |
| | GGCAC |
| pBR-GR-recN4 | attgctgaccaccagccagaatgcattggcatt |
| | aatggccgacggtgaagCCGATACGCGAGCGAA |
| | CGTGA |
| recN-WII-1 | cagtcacgacgttgtaaaacgacggccagtgaa |
| | tccgtaatcatggtcatAGTCGTTTTCCTGTAT |
| | GAAAAAC |
| recN-WII-2 | cgcggtatggcatgataccgcccggaagagagt |
| | caattcagggtggtgaaTTACCAATGCTTAATC |
| | AGTGAG |
| recN-plan1-A | aaacagatcgaagaaggggttgaatcgcaggct |
| | attctggtggccggaaggTTACACTCTCCATTC |
| | CATCAG |
| Pt5-recN-1 | atgagacaataaccctgataaatgcttcaataa |
| | tattgaaaaaggaagagtAAATCATAAAAAATT |
| | TATTTG |
| P_{recN}-planC-1 | gagcggataacaatttcacacagaattcattaa |
| | agaggagaaattaaccatATGAGTATTCAACAT |
| | TTCCG |
| P_{recN}-bla-1 | aaacagatcgaagaaggggttgaatcgcaggct |
| | aaactggtggccggaagGCGAAGCGGCATGCAT |
| | TTACG |
| bglF-bla1 | cgggatcatggtgtcgtagcccagcacggtgaa |
| | gttattgatacacagcgAAATCATAAAAAATTT |
| | ATTTG |
| bglF-bla2 | acaccgcggggaaacgcttcggcggtaatccat |
| | ttactgccatggtgattTTACCAATGCTTAATC |
| | AGTG |

The procedures to construct P_{recN}′-'lacZ reporter fusion gene are described below. In the first step pBR322 was used as a template for PCR using oligonucleotides, pBR-GR-recN1 and pBR-GR-recN4, see Table 12, to generate a DNA fragment containing bla, rop and ori. The regions in uppercase were responsible for PCR amplification and the regions in lowercase were homologous to the regions flanking the recN promoter locus (AE000347 bases 5433-5516) of E. coli MG1655. The PCR product was cloned into pBR322 using the method of Lee et al, 2001, “A highly efficient Escherichia coli-based chromosome engineering system adapted for recombinogenic targeting and subcloning of BAC DNA,” Genomics 73: 56-65, resulting in plasmid, pCM105, which contained recN promoter in the opposite orientation of bla (P_{recN}-bla cassette). In the second step of reporter strain construction, the P_{recN}-bla cassette was amplified by PCR using pCM105 as the template and primers, recN-WII-1 and “recN-WII-2, see Table 12. The PCR fragment was then inserted into the lacZ locus by homologous recombination as described in Datsenko and Wanner, 2000. See U.S. Pat. No. 6,355,412 and U.S. Pat. No. 6,509,156. The regions of these primers in upper case were responsible for PCR amplification of the P_{recN}-bla cassette while the regions in lower case were homologous to regions flanking the lacIZ locus so that they could mediate the recombination at that locus. In the resulting strain, CM203, both lacI and the region between lacI and lacZ were replaced by the P_{recN}-bla cassette and the expression of lacZ was under the control of the recN promoter. Next, the bla gene of the P_{recN}-bla cassette contained in CM203 was replaced by the multiple drug resistance cassette (P_{T5} aph(2″)-Ib-aac(6′)-Im), resulting in strain CM212. Using CM191 as template, the (P_{T5} aph(2″)-Ib-aac(6′)-Im) cassette was amplified using the following oligonucleotide primers, recN-plan1-A and Pt5-recN-1, see Table 12. These primers also contained regions homologous to the bla gene so that they could mediate the homologous recombination to replace the bla gene with the P_{T5} aph(2″)-Ib-aac(6′)-Im. The regions of these primers in upper case were responsible for PCR amplification of the P_{T5} aph(2″)-Ib-aac(6′)-Im cassette while the regions in lower case were homologous to bla gene.

In order to obtain the bla gene under the control of the T5 promoter, the aph(2″)-Ib-aac(6′)-Im resistant genes in the P_{T5} aph(2″)-Ib-aac(6′)-Im cassette in CM212 were replaced with bla. Using CM191 as the template, bla was amplified with the oligonucleotide primers, P_{recN}-planC-1 and P_{recN}-bla-1, see Table 12. The sequences in uppercase were for PCR amplification and the sequences in lower case were for homologous recombination. The PCR fragment was introduced into CM212 and recombined at the targeting locus, resulting in the replacement of the aph(2″)-Ib-aac(6′)-Im drug-resistant genes with bla gene. The resulting strain, CM225, contains the (P_{T5} bla) cassette in which the expression of bla was under the control of the T5 promoter.

Finally, the (P_{T5} bla) cassette was amplified from CM225 and inserted into the bglF locus (AE000449 bases 5104-6981, also listed as bglC=M15746, into the intermediate strain CM212 by homologous recombination, resulting in screening strain CM242. See Datsenko and Wanner, 2000; U.S. Pat. No. 6,355,412; U.S. Pat. No. 6,509,156. The following oligonucleotide primers, bglF-bla1 and bglF-bla2, see Table 12, were used for both PCR amplification and the bglF locus disruption. Sequences shown in upper case were for PCR amplification of the P_{T5} bla cassette while the sequences in lower case shared homology with the bglF locus. T5 promoter was in the opposite orientation to bglF gene.

Additionally, the ilvG::cat allele was introduced into each of the strains having lacZ reporter fusion genes as described above (CM191 and CM242) to render the resulting strain the auxotrophic phenotype (dependent upon Ile and Val) by standard P1 phage transduction.

The level of drug resistance of these screening strains (both intermediates and finals) was evaluated by standard MIC assay in MHB liquid medium and the result of Strain CM242 is summarized in Table 12.

TABLE 12
Minimal inhibitory concentrations (MIC₉₀) of antibiotics
against E. coli MG1655 and CM242 in MHB medium.

| Antibiotic | MG1655 | CM242 |
|---|---|---|
| Actinomycin C | 50 | 200 |
| Actinomycin D | 200 | 200 |
| Ampicillin | 1.6-3.2 | >400 |
| Apramycin | 3.2-6.3 | 400 |
| Aztreonam | 0.1 | 0.3 |
| Bleomycin | ≦0.2-0.4 | 200 |
| Chloramphenicol | 3.2 | 100 |
| Coumermycin A1 | 12.5 | 6.3 |
| Daunorubicin | 200 | 100 |
| Dibekacin | 3.2 | 400 |
| Erythromycin | 25 | 25 |
| Gentamicin | 0.8 | 100 |
| Kanamycin | 0.8-1.6 | >400 |
| Mitomycin C | 0.8 | 3.2 |
| Nalidixic Acid | 6.3 | >400 |
| Neomycin | 0.8 | 50 |
| Netilmycin | 1.6 | 200 |
| Spectinomycin | 6.3-12.5 | >400 |
| Streptomycin | 3.2 | >400 |
| Streptonigrin | 3.2 | 3.2 |
| Streptothricin | 0.4-1.6 | >400 |
| Tetracycline | 0.4-3.2 | >400 |
| Tobramycin | 0.4 | 100 |
| Trimethoprim | ≦0.4 | >400 |

The responses to DNA damaging agents of the screening strains described above were measured by analyzing the β-galactosidase activity in the presence of DNA damaging drugs. This method is described in detail in “A Short Course in Bacterial Genetics” 1992, ed. Miller, Cold Spring Harbor Press, pp. 72-74. The P_{sulA}′-'lacZ fusion gene in CM191 was induced 15 fold compared to a non-induced control two hours after the addition of mitomycin C (0.5 μg/ml). In the case of CM242 which contains the P_{recN}′-'lacZ fusion gene, a more than 40-fold induction had been observed. Such induction was also drug-specific. In an experiment when phosphomycin (at 10 μg/ml) was used, there was no induction of the β-galactosidase activity.

The screening strains' responses to DNA damaging agents were further confirmed by S-Gal staining assay. When mitomycin C and streptonigrin (both were well-known DNA damaging drugs) and S-Gal, the indicator, were applied to the screening strains growing on solid medium, blue-colored rings were visible around colonies in drug-impregnated disks. The lacZ reporter gene was not expressed when non-DNA damaging drugs, such as Nalidixic acid, trimethoprim, were present, while its expression was induced by the presence of mitomycin C, streptonigrin, and other DNA damaging drugs. Actinomycin D was an exception. CM242 was not sensitive to this drug, although it disrupts DNA structure. PETG (2-phenyethyl B-D-thiogalactoside, Sigma, St Louis Mo.), was used in this experiment to suppress unspecific background activity.

5.3.1.e. E. coli Screening Strain CM435: Increased Drug Permeability

The screening strain CM435 is a derivative of CM400 (a screening strain constructed by the inventors, see details in Table 4), which bears identical drug resistance markers as those of CM400 but further contains a tolC null mutation, ΔtolC:: (P_{T5}aph(2″)-Ib-aac(6′)-Im). The tolC gene product is an outer membrane protein and its null-mutation increases the permeability of the outer membrane of E. coli. Thus, CM435 is hypersensitive to certain classes of antibiotics but maintains the overall multiple drug resistant phenotype of CM400. Another strategy to establish screening strains with increased permeability of drugs is to introduce mutations that reduce the efflux of the drugs from the cell. The procedure of establishing screening strain CM435 is described as follows:

Resistance markers were introduced by either transducing already existing resistance alleles present at chromosomal loci in other E. coli strains (nalA37, rpsL150, metE::tetA) or they were introduced (for instance, Tn7-associated genes, aph(2″)-Ib, aac(6′)-Im, TEM-1 bla and aac(3′)-IV), Tn5-associated genes) using the method described in Datsenko and Wanner, 2000. See U.S. Pat. No. 6,355,412 ans U.S. Pat. No. 6,509,156. Chromosomal targets were chosen that had been shown to be non-essential (citAB-operon, csgA, λ-attachment site, bglF-operon) or leading to an auxotrophy for methionine (metE) or resulting in additional resistance to compounds using the ferrichrome uptake system (fhuA).

The entire cassette including the T5 promoter (P_{T5}aph(2″)-Ib-aac(6′)-Im) was amplified using pCM100 (an intermediate screening strain bearing the aforementioned cassette) as the template and the fragment was then used to replace chromosomal tolC by the homologous recombination method described by Datsenko and Wanner. See U.S. Pat. No. 6,355,412 and U.S. Pat. No. 6,509,156. The following oligonucleotide primers were used for the tolC disruption: “tolC-aph1” 5′-cgcgctaaatactgcttcaccacaaggaatgcaaatgaagaaattgaccAAATCATAAAAAATTTATTTG-3′ and “tolC-aph2” 5′-cgaagccccgtcgtcgtcatcagttacggaaagggttatgaccgttactg TTACACTCTCCATTCCATCAG-3′. The regions of these primers in uppercase hybridize to the multiple drug resistant cassette while the regions in lower case are homologous to sequences nearby tolC locus, therefore mediating the insertion of the cassette into the tolC locus (in the same orientation as the tolC gene).

Screening CM435 was constructed by further delivering other multiple drug resistant genes, which are derived from MG1655 (see section 5.3.1.a), into the aforementioned strain bearing tolC mutation by P1 phage mediated gene transduction (see details in section 5.3.1.a).

The level of drug resistance of screening strains CM400 and CM435 was evaluated by standard MIC assay in MHB liquid medium and the results are summarized in Table 13. According to the MIC results, CM435 is hypersensitive to Bleomycin, Daunorucibin, Coumermycin A1 and Erythromycin. Screening strain CM435 and others, which are hypersensitive to certain types of antimicrobial agents, are advantageous in detecting bioactive compounds with low concentration in the screening system.

TABLE 13
Minimal inhibitory concentrations (MIC₉₀) of antibiotics
against E. coli MG1655 CM400, and CM435 in MHB medium.

| Antibiotic | MG1655 | CM400 | CM435 |
|---|---|---|---|
| Actinomycin C | 256 | >256 | >256 |
| Actinomycin D | 256 | 256 | 256 |
| Ampicillin | 8 | >64 | >64 |
| Apramycin | 4 | 64 | >64 |
| Aztreonam | 0.06 | <0.06 | 0.125 |
| Bleomycin | 2 | >64 | 8 |
| Chloramphenicol | 8 | >64 | >64 |
| Coumermycin A1 | >64 | 64 | 4 |
| Daunorubicin | >256 | >256 | 8 |
| Erythromycin | >64 | 64 | 4 |
| Gentamicin | 1 | >64 | >64 |
| Kanamycin | 8 | >64 | >64 |
| Nalidixic Acid | 8 | >64 | >64 |
| Neomycin | 2 | 16 | 8 |
| Netilmycin | 0.5 | >64 | 64 |
| Phosphomycin | 32 | 8 | 64 |
| Rifamycin | 16 | >64 | 16 |
| Spectinomycin | 16 | >64 | >64 |
| Streptomycin | 4 | >64 | >64 |
| Streptonigrin | 8 | 8 | 0.5 |
| Streptothricin | 2 | >64 | 64 |
| Tetracycline | 2 | >64 | 64 |
| Trimethoprim | 0.5 | >64 | >64 |

5.3.1.f. Gram-Positive Screening Strains: Multiple Drug Resistances

Applying an approach analogous to that taken to obtain the multiple resistant E. coli strains, a Gram-positive organism can be modified to exhibit a similar antibiotic resistance profile. Several Gram-positive strains (e.g. Staphylococcus aureus or Enterococcus faecalis) are available that are resistant to multiple drugs (nalidixic acid, apramycin, gentamycin, kanamycin, neomycin, spectinomycin, streptomycin, tobramycin, erythromycin, trimethoprim). Additional drug resistant genes of interest and their sources of availability are listed in Table 14. The screening strain with additional drug resistance can be constructed according to the following procedures.

TABLE 14
Relevant resistance markers

| Drug resistance | Gene | Source | Supplier | GenBank # |
|---|---|---|---|---|
| tetracycline | tetA(M) | pGO533 | S. Projan* | M21136 |
| Ampicillin | pbp5† | E. faecalis | Available in | X78425 |
| | blaZ | S. aureus | almost all | AY373761 |
| | | plasmid | research labs | |
| chloramphenicol | cat | pIP501 | T. Horaud** | X65462 |
| vancomycin | vanA | Tn1546 | Arthur et al.*** | M97297 |

*Nesin et al., 1990, Antimicrob Agents Chemother, 34: 2273-2276.
**Trieu-Cuot et al., 1992, Plasmid, 28: 272-276.
***Arthur at al., 1993, J Bacteriol. 175 (1): 117-127
†Overproduction of pbp5 results in resistance to β-lactams. Fontana et al., 1983, J Bacteriol 155(3): 1343-50 and Arthur et al., 1993, J Bacteriol 175(1): 117-127.

The E. faecalis strain V583 has been sequenced (Genbank accession # NC_{—}004668) and can be used as the parent strain for the introduction of additional drug resistance genes. The following two strategies can be taken. First, resistance markers can be introduced in the genome using protocols for gene disruption mutagenesis. Briefly, flanking regions of a non-essential region of the Enterococcus genome are amplified by PCR, ligated and then sub-cloned into an E. coli-Enterococcus shuttle vector such as pAM401ts. Weaver, et al., 1998, “Isolation of a derivative of Escherichia coli-Enterococcus faecalis shuttle vector pAM401 temperature sensitive for maintenance in E. faecalis and its use in evaluating the mechanism of pAD1 par-dependent plasmid stabilization,” Plasmid, 40(3):225-32. The resulting plasmid construct can then be transformed into the Enterococcus recipient strain by standard electroporation. The single-crossover mutants can be obtained at permissive temperature (30° C.). After a shift to a non-permissive temperature (38° C.), mutants containing double-crossovers can be recovered. Target sites for the resistance cassettes can be any non-essential region of the genome but it is preferable to target known genes to generate auxotrophies (e.g. pyrC; Li et al., 1995, “Generation of auxotrophic mutants of Enterococcus faecalis,” J Bacteriol 177: 6866-73) or genes involved in enterococcal virulence (e.g. fsrB; Qin, et al., 2001, “Characterization of fsr, a regulator controlling expression of gelatinase and serine protease in Enterococcus faecalis OG1RF,” J. Bacteriol. 183(11):3372-82).

The drug-resistant genes introduced are not limited to those listed in Table 13, and are decided by the type of compound sought. Other genetic traits, such as reporter fusion genes, auxotrophic mutations, sensitivities to certain physiological conditions, etc., can all be incorporated into the Gram-positive screening strain in a manner similar to that of constructing E. coli. screening strains as described above. What genetic traits are desired depends on the type of compounds sought. All commonly used gene transfer/genetic engineering methods, such as phage-mediated transduction, plasmid transformation, homologous recombination, etc. are all applicable in establishing Gram-positive bacterial screening strains as known by the skilled in the art.

5.3.2. A Method for Selecting Microorganisms Producing Erythromycin and Vancomycin From Pooled Soil Samples.

An advantage of the macrodroplet screening system with antibiotic resistant screening strains is that millions of actinomycetes can be screened in a single year. In the past, only tens of thousands of actinomycete strains were typically screened by pharmaceutical companies per year. Therefore, antibiotic producing actinomycetes present in soil a low frequencies can be found with this high throughput screening system. To test this concept, thousands of soils were pooled and actinomycete spores were isolated from the pool. Millions of spores were plated on an agar based medium containing 1 mg/ml of vancomycin or erythromycin. The producers of these antibiotics should be resistant to these concentrations of antibiotics based upon their genetic resistance encoded by VanA and ErmE antibiotic resistances, respectively, and most actinomycetes should not grow in the presence of these antibiotics. Several producers of the target antibiotic were isolated. Production of the both erythromycin and vancomycin were confirmed by LC-MS on putative peaks of the antibiotics in fermentation broths shown previously to be active against susceptible S. aureus. For erythromycin, 1 producer was discovered per 275,000 actinomycetes. The first 500 base pairs of the 16S rDNA gene of 3 different producers isolated were sequenced which resulted in no matches to Saccharopolyspora erythraea. The extent of differences in nucleotides of these strains to sequences of strains deposited in GENBANK indicated the novelty of the strains isolated. For vancomycin, 1 producer was discovered per 24,000 actinomycetes. The sequence of the 16S rDNA gene of all of the isolates producing vancomycin matched the reference sequence of Amycolatopsis orientalis. This experiment showed that screening millions of actinomycetes from pooled soil samples readily led to the isolation of producers of vancomycin and erythromycin which are not commonly isolated in standard low throughput screens.

5.3.3. A Method of Screening Antibacterial Agents Using CM166 as the Screening Strain.

This example describes the detailed screening procedures for microorganisms producing antibacterial compounds from soil samples, using CM166 as the screening strain. The microorganism sources can be derived from other environmental samples as well.

Soil samples collected throughout the United States, Canada and the UK were pooled, microorganisms extracted in a way that enriches for actinomycetes and the spores stored. In detail, soil samples were heat-treated at 60° C. for 1 hour in order to reduce the number of non-actinomycete bacteria. Three-hundred milliliters (300 ml) Na Cholic acid/PEG8000 solution and 15 g chelex 100 was added to each 150 g soil in pot. The soil samples were shaken at 220 rpm at 5° C. for 2 hours and then centrifuged at 2250 rpm for 1 min at 5° C. The supernatant was collected (Sup 1) and the pellet was resuspended with 200 ml Na Cholic acid/PEG solution and the procedures of shaking and centrifugation were repeated once. The resulting supernatant (Sup 2) was collected and pooled with Sup 1, centrifuged at 3000 rpm at 5° C. for 30 minutes. The pellet generated was agitated and suspended in 10% glycerol, 0.1% Tween 80 solution (50 ml for 2 pots). One milliliter (ml) of the solution containing the suspended pellet was serial diluted to 10⁻⁵ using PBS as the diluent. The diluted samples were first filtered through a 5 μm filter to eliminate some larger spored fungi or fungal mycelial fragments and then 100 μl of each of the 10⁻² to 10⁻⁵ dilution samples were plated out with 30 μg/ml nalidixic acid, 40 μg/ml trimethoprin, 50 μg/ml nystatin, and 50 μg/ml cycloheximide. The rest of the resuspended pellet samples were aliquoted and stored at −135° C.

Next, microorganisms from the soil samples collected as described above were encapsulated to form macrodroplets populated by a single microorganism in each bead, according to the pre-determined cfu count of the particular soil extract. In this process, an aliquot of the soil bacteria suspension was diluted in 10% glycerol to generate an inoculum of the required density. This adjusted suspension was then mixed with nutrient medium, counterselective agents (see above) and 1.4% sodium alginate and the resultant mixture processed through an Inotech Encapsulator® Research device, to produce gel-beads (called macrodroplets) by the formation of droplets of liquid from a fluid stream. The droplets solidified into gel-beads as they came into contact with a calcium chloride bath, which also contained nutrient medium and counterselective agents. After a defined hardening time the macrodroplets were washed to remove excess calcium chloride and to stop the hardening process. Smidsrod and Skjak-Braek, 1990, “Alginate as immobilization matrix for cells,” Trends Biotechnol. 8(3):71-8. Cured macrodroplets containing microorganisms were then spread in dishes from which excess fluid was removed. The macrodroplets subsequently allowed growth of the target organisms and production of secondary metabolites such as anti-microbial agents. Macrodroplets were spread evenly over the plate using a sterile spreader (5,000 particles per plate) and incubated at 28-30° C. for 7 days to allow the growth of the microorganism within, and the production of secondary metabolites.

CM166 (see section 5.3.1.b for details) was used as the test strain in this example. Two to three single colonies of CM166 were inoculated into a shake flask containing 10 ml of MHB medium with 40 μg/ml Tmp and 200 μg/ml Amp. After the cells were cultured at 35° C. for 2-3 hours, an OD reading was performed. If the OD reading was above 0.1 (equivalent to 2×10⁸ cfu), the screening strain was ready for dilution. Otherwise, the strain needed to be incubated for longer time until the OD read reached >0.1. Medium for the macrodroplet assay (in 1 L Duran, add 10.5 g MHB power, 4 g agar (0.8%), and 1 g DNA) was autoclaved and cooled to ˜45° C., and ampicillin (25 mg/ml), tetracycline (10 mg/ml), daptomycin (20 mg/ml) and biotin (0.1 μg/ml) were added, together with 2.5 ml screening strain. The medium containing screening strains as described above was poured on top of the macrodroplets, which had been growing for 7 days at 28-30° C. as described above. The plates were preincubated at 4° C. for 30 minutes to allow the active agents from the macrodroplets to diffuse out into the surrounding media before the screening strain began growing. The plates were incubated at 37° C. overnight. A clear zone is observed around macrodroplets that produce antibacterial agents. The addition of DNA to the medium can reduce the screening background due to non-specific DNA damaging agents. Another useful procedure was to include biotin in the screening medium to reduce background activity due to actinomycetes that produce synergistic mixtures of biotin-scavenging proteins and biotin antagonists such as streptavidin and stravidin respectively. The addition of DNA samples in the screening medium was omitted when DNA damaging agents were the target compounds.

A method for recovering actinomytes from macrodroplets is generally described in section 5.2.3.5. In this example, macrodroplets producing zones of inhibition were collected manually, by sterile Transfertube® (Spectrum Laboratories, Inc.), or by another appropriate method such as an aspirator, and each individual macrodroplet was placed in a separate well of a 96-well 0.22 μm filter MultiScreen-GV (Millipore) plate. 150 μg/ml of a solution containing 10% glycerol, 0.1% Tween 80 and 20 μg/ml aztreonam was added to each occupied well of the filter plate. Using the MultiScreen vacuum manifold (Millipore), the macrodroplets were washed at least twice. The wash solution was vacuumed through the filter of the 96-well plate as waste, leaving the macrodroplets intact in the wells of the MultiScreen filter plate. 150 μl of 0.25% sodium citrate and 20 μg/ml aztreonam was added to each well and the MultiScreen filter plate was shaken at 750 rpm for at least 30 minutes to dissolve the macrodroplet. The wells of the filter plate were then washed at least twice using the MultiScreen vacuum manifold (Millipore), leaving each biomass in a liquid-free well of the 96-well plate. A sterile, 0.22 μm cellulose nitrate membrane filter (Whatman) was aseptically transferred to each well of a 6-well plate containing growth media, primarily media identical to that used for macrodroplet production. Each biomass was aseptically transferred onto a separate membrane filter in the E-well plate using sterile toothpicks. The microorganisms on the membrane, actinomycetes in this example, were allowed to grow at 28° C. for 7 days.

Phenotypically differentiated colonies with obviously different appearances were picked, plated on an oatmeal-based medium in 6-well plates and incubated at 28° C. for 5-7 days. Colonies with identical phenotypes were de-replicated (pick one colony among several identical ones). Fermented strains were collected. An aliquot was inactivated at 90° C. for at least 20 minutes and then submitted for 16S rRNA gene sequencing. The strains were further fermented and active compounds were purified and characterized.

Several actinomycete strains were selected using CM166 as the screening strain, and further chemical characterization indicated that the bioactive compounds were thiolutin, aureothricin, lydimycin, albomycin, streptavidin and stravidin-like compounds. See Table 4. No antibacterial agents whose resistant genes were incorporated into the CM166 genome were selected by this screening method.

5.3.4. A Method of Screening Anti-Fungal Agents Using Candida Strains as the Screening Strains

This example illustrates the screening procedures for antifungal agents using multiple drug resistant Candida strains as the screening strains. Although S. cerevisiae, as a well-established experimental system in biomedical research, is much easier to manipulate, we choose Candida as an example in this application because Candida strains are known pathogens for human beings and they cause systemic infections, which usually are life-threatening.

Candida strains (eg. C. albicans, C. glabrata, C. krusei, etc.) bearing multiple drug resistances are established by isolation from clinical sources, high-dose drug-treatment, mutagen-treatment or genetic engineering. Clinical sources for multi-drug resistant Candida strains are collected from patients who had been administrated with anti-fungal drugs for extended periods of time and the methods of isolating these strains are well known in the art. See Dick et al., 1980, “Incidence of polyene-resistant yeasts recovered from clinical specimens,” Antimicrob. Agents Chemother. 18(1):158-63; Dassanayake et al., 2002, “Molecular heterogeneity of fluconazole-resistant and -susceptible oral Candida albicans isolated within a single geographic locale,” APMIS, 110(4):315-24. Such drug-resistant strains also can be generated by multiple rounds of high-dose drug selection. The advantage of doing drug selection is that one can select Candida strains containing desired drug resistant profile in accordance with the type of compounds sought. In addition, Candida screening strains can be established by genetic engineering. As discussed in section 5.2.2.2, genetic basis of antifungal drug resistance (e.g. resistant to polyenes and azoles) are revealed and one can introduce these mutations into the recipient Candida strains to generate multi-drug resistant strains, which serves as the screening strains for antifungal compounds.

The procedures for soil sample collection, extraction and actinomycetes enrichment are exactly the same as described in section 5.2.3.1. The subsequent macrodroplet bioassay is also similar. Singly populated macrodroplets are generated and cultured in vitro for 5-10 days, but most frequently 7 days. Screening strains are prepared according to the following procedure. Colonies of Candida screening strains are inoculated into shaking flasks with a suitable medium including selective antifungals and counter-selective agents (see section 5.2.3). The optimal medium condition varies from strain to strain and is discussed in detail in Moore & McMullan, 2003, “Comparison of media for optimal recovery of Candida albicans and Candida glabrata from blood culture,” Ir. J. Med. Sci. 172(2):60-2. Generally, TSBYE is the optimal medium for both C. albicans and C. glabrata. After culturing the Candida strains overnight, their cell density is determined by O.D. The optimal O.D. can be determined by culturing Candida screening strains with different O.D. read for 48-72 hours and pick the one which allows the growth of a single layer of Candida strains on an agar plate. After the optimal O.D. read is determined, Candida culture with the optimal O.D. read is mixed with TSBYE having counterselective drugs, as well as commonly used antifungal drugs, such as amphotericin B, candicidin, pimaricin, nystatin, etc. The medium containing Candida screening strains are poured on top of the macrodroplets, which have been growing for 5-10 days at 28-30° C. See details in section 5.2.3. The plates are placed into an incubator at 30° C. for 48-72 hours until a single layer of Candida cells are clearly seen. A clear zone will be observed around a positive macrodroplet, which produces antifungal agents.

The recovery of microorganisms from beads producing zones of antifungal activity and their culture and dereplication is as described in 5.2.3.5 except that an antifungal drug to which the screening strain is sensitive is required in the clean-up procedure.

5.3.5. A Method of Screening Anti-Neoplastic Agents Using a Human Tumor Cell Line as the Screening Strain.

In addition to be the principal source for anti-bacterial agents, microorganisms, in particular actinomycetes, have also been the source of a number of important anti-tumour drugs. Examples include DNA-interactive agents, such as bleomycins, dactinomycin, mitomycin C and anthracyclines, as well as compounds with different modes of action, for example, epothilones (produced by myxobacteria, not actinomycetes), which act by stabilizing microtubules during cell division. Höfle et al., 1996, “Epothilone A and B—Novel 16-Membered Macrolides with Cytotoxic Activity Isolation, Crystal Structure, and Conformation in Solution,” Angew. Chem. Int. Ed. Engl. 35: pp. 1567-1569.

Human tumor cells can also be used as the screening strain in the macrodroplet screening assay (see section 5.2.3) for the detection of compounds with anti-neoplastic activities or detection of cytotoxic compounds produced by bacteria growing in macrodroplets by applying a two-layer agar diffusion method illustrated in FIG. 2. Similar bioassays, which have been used extensively to screen and monitor the safety of biomaterials used in manufacturing medical devices, are also applicable in our high-throughput drug screening system seeking for anti-tumor drugs, for example, the method discussed in Rosenbleuth et al., 1965, “Tissue culture method for screening toxicity of plastics to be used in medical practice,” J. Pharm. Soc. 54:156.

In this example, HepG2 cells derived from a human hepatocellular carcinoma (obtained from the ECACC) are used as the test cell line (the screening strain) for macrodroplet bioassays. The advantage of using this cell line is that it has a higher resistance to toxic compounds than many non-hepatic cell lines; therefore, anti-tumor drugs with high potency may be discovered by screening against this cell line. The drug-screening can be achieved by the following procedures illustrated schematically in FIG. 2. Cells are grown in Eagles Minimal Essential Medium (EMEM) supplemented with 10% fetal bovine serum and incubated at 37° C. in a humidified atmosphere with 5% CO₂ in air. Cultures are passaged in antibiotic-free medium and are re-initiated from frozen stocks at 3-month intervals.

Macrodroplets containing actinomycetes are prepared as previously described and incubated in 20×20 cm bioassay plates for 7 days at 28° C. FIG. 2, item 200. After this period the macrodroplets, are embedded in a layer of agar (0.6% w/v agar in EMEM medium) and the plates allowed to set at 4° C. Id., item 210. Plates are then overlayed with 0.8% (w/v) low melting point agarose in EMEM (cooled to 37° C.), which has been inoculated with HepG2 cells to a density of 2×10⁶ cells/ml. Id. Item 220. Plates are kept at 4° C. for 15 min to allow the agarose to set before being incubated in a cell culture incubator for 48 hours. Areas of cytotoxicity are visualized by overlayed the agarose surface with a 5 mg/ml solution of 3-(4,5-dimethylthiazol-2-yl)-2,5-diphenyltetrazolium bromide (MTT) in growth medium, and incubating at 37° C. for 2 hours. Viable cells are stained purple by the production of reduced formazan, while areas of toxicity are observed as zones of clearing. Id. item 230.

Macrodroplets producing cytotoxic compounds are subsequently recovered, microorganisms contained inside positive macrodroplets are fermented in a larger volume and dereplicated by phylogenetic characterization by methods commonly known in the art, for example, 16S rDNA gene sequencing. The compounds causing cell cytotoxicity are purified and their cytotoxic properties are further characterized. Those which induce cytotoxocity by causing DNA damage, inhibiting cell division, etc., are potential anti-tumor drug candidates.

5.3.6. A Method for Screening DNA-Interactive Anti-Neoplastic Agents Using a Sensitized Bacterial Screening Strain.

Historically, DNA represents the end-target for many cytotoxic agents used in a clinical setting. Examples include dactinomycin, mitomycin C, bleomycin, daunorubicin. Anthony and Twelves, 2001, “DNA: Still a target worth looking at? A review of new DNA-interactive agents,” Am. J. Pharmacogenomics. 1: 67-81. Because tumor cells have lost control of cell division and are actively replicating their DNA, they are far more sensitive to DNA damaging drugs. Therefore, compounds causing DNA damage are potential antitumor drug candidates. Although they might be used clinically as chemotherapeutic agents in the treatment of cancer, most DNA-interactive agents also have significant anti-bacterial activity. This example illustrates the use of a genetically modified bacterial screening strain DR1212 (see details in section 5.3.1.c), which is highly sensitive to DNA damaging agents, to screen for microorganisms, especially actinomycetes, producing DNA-interactive compounds with potential anti-tumor potency.

Most bacteria survive damage to cellular DNA because they have an effective DNA repair system. RecA, a major component of the SOS response system, plays an essential role in both DNA repair and the up-regulation of the DNA repair system when the cell is experiencing stress, such as UV radiation or the exposure under DNA damaging agents. As described in section 5.3.1.c, the screening strain DR1212 has a mutated recA locus in which a cat gene, encoding chloramphenicol acetyltransferase, is inserted. The disruption of the recA locus makes DR1212 hypersensitive to DNA damaging agents.

Screening for DNA-interacting agents was carried out using the standard macrodroplet screening method previously described, with the exception that DNA was excluded from the bioassay medium. Following overnight incubation at 35° C., macrodroplet beads from zones of inhibition are recovered and processed via the normal route as discussed in section 5.2.2.4.

Alternatively, a second round screening was performed to increase specificity of the screening. Microorganisms obtained as the outcome of the screening described above were subject to a second round of screening using screening strains having a lacZ reporter gene controlled by promoters responsive to DNA damage, such as CM191 and CM242 (see section 5.3.1.d. The details of screening procedure are discussed in both section 5.2.3. Microorganisms from macrodroplets producing zones of activity were purified using methods previously described and fermented in larger volumes. Their species specificities were determined by sequencing their 16S rRNA genes and the active compounds were purified and further characterized. Several active compounds were selected by DR1212, such as chrysomycin, streptonigrin, daunomycin and chromomycin, which are all known DNA damaging agents. See Table 4.

5.3.7. A Method for Screening Anti-Neoplastic Agents Using Polyene-Resistant Yeast as the Screening strain.

This example illustrates the use of the macrodroplet screening system to detect cytotoxic compounds with potential anti-neoplastic activity using polyene-resistant yeast strain as the screening organism.

Yeasts are being used increasingly as model organisms for anti-tumor drug discovery. This is partly due to the inherent tractability of yeast cells compared to mammalian cell lines and their adaptability to different assay systems. In addition, comparison of the genomes of both humans and yeast cells have shown a high degree of conservation in the major signaling proteins and basic cellular processes between mammalian cells and lower eukaryotic systems such as yeast. Ma et al., 2001, “Applications of yeast in drug discovery,” Prog. Drug Res. 57:117-162.

Yeast cells are also amenable to genetic manipulations. As eukaryotic cells, they are more suitable as hosts for the expression of specific human genes, e.g. potential drug targets, allowing more target-orientated bioassays to be developed. Munder & Hinnen, 1999, “Yeast cells as tools for target-oriented screening,” Appl. Microbiol. Biotechnol. 52:311-320. These factors give yeast cells a distinct advantage over prokaryotic bacteria or mammalian cell lines as screening organisms for the discovery of compounds with potential cytotoxicity in human cells.

The yeast strain commonly used as a model for mammalian cytotoxicity studies is Saccharomyces cerevisiae. As this yeast is unicellular with similar growth conditions to bacteria, it can be easily implemented into the macrodroplet bioassay, replacing E. coli as the screening strain. The bioassay is otherwise carried out analysis of ketoconazole resistant mutants of Saccharomyces cerevisiae. Watson, et al., “Isolation and analysis of ketoconazole resistant mutants of Sacharomyces cerecisiae,” 1990, J. Med. Vet. Mycol. 26:153-162. The use of such a strain as the screening organism would reduce the likelihood of selecting already known antifungal drugs. Other yeast strains with multiple drug resistance are also suitable to serve as the screening strain in this assay. These strains can be either established by the treatment of high doses of antifungal drugs, or by genetic engineering, delivering antifungal drug resistant genes into the recipient yeast cells. See details in section 5.2.2.1.

5.3.8. A Method for Screening Antiviral Agents by Selecting Natural Compounds which Inhibits the Entry of HIV-1 into Host Human CD4 Lymphocytes.

This example teaches the efficient, ultra-high throughput screening of natural compounds that disrupts the interaction between HIV-1 gp120 protein and CD4 receptor of human CD4 T lymphocytes, thus blocking the entry of HIV-1 into its host cells.

HIV-1 enters into its host cell, human CD4 T lymphocyte via the formation of an anchoring complex and both gp120 and CD4 are the major players for the entry of HIV-1 into its host cells. Other cell surface co-receptors and viral envelop proteins also facilitate this process. See Fields, et al., 2001, “Virology.” Also see Ruibal-Ares et al., 2004. The disruption of the formation of such anchoring complex blocks the entry of HIV-1, thus preventing HIV-1 infection. Schols, 2004.

In this example, we use S. cerevisiae as the screening strain. Cell lines are suitable screening strains applicable in this method as well. Bacterial strains may also work. The mechanism of how this method works is discussed in section 5.2.2.1. The screening strain exhibits multiple drug resistances and auxotrophies. In addition, three plasmids are introduced into the screening strain. Plasmid A contains a lacZ reporter gene under the control of bacterial LexA operator so that the expression of lacZ is dependent upon the presence of LexA protein, which consists of two functional domains, DNA binding domain (DBD) and activation domain (AD). The association of DBD and AD forms a functional transcriptional factor driven the lacZ reporter gene expression. Plasmid B contains an expression cassette of LexA-DBD-gp120 fusion protein. Plasmid C expresses the LexA-AD-CD4 fusion protein. The interaction between gp120 and CD4 results in the association of DBD and AD, which consequently results in the turning-on of the lacZ reporter gene. However, compounds which disrupt the interaction between gp120 and CD4, also destroy the association of DBD and AD, and consequently turn off the lacZ reporter gene expression.

In addition to gp120, other HIV-1 envelop proteins involved in the virus entry, such as gp41, also can be the target to construct fusion proteins as described above in this section. Liu & Jiang, 2004, “High throughput screening and characterization of HIB-1 entry inhibitors targeting gp41, theories and techniques,” Curr. Pharm. Des., 10(15):1827-43. Similarly, other cell surface receptors involved in the formation of the entry complex, such as chemokine receptors, CXCR4 and CCRS, are desired targets as well.

The screening procedure is the same as discussed in section 5.2.3. In general, soil samples are collected, pooled and microorganisms are extracted and cultured in vitro in a condition that actinomycetes are enriched. The enrichment procedure is skipped if no specific microorganisms are targeted. Single cells or spores are encapsulated to form macrodroplets and further cultured in vitro under the preferred conditions as described in section 5.2.3. S. cerevisiae screening strains are cultured under the condition well-known in the art and mixed with semi-solid medium, then overlayed on top of the macrodroplets. The positive macrodroplets are detected by in situ staining with S-gal. Screening strains surrounding negative macrodroplets are blue, whereas screening strains surrounding positive macrodroplets are white.

Microorganisms contained in the positive macrodroplets are recovered, further fermented, and their species determined by 16S rDNA gene sequencing as described in section 5.2.3.5. Active compounds are purified from cell extract and their chemical properties are further characterized.

Each patent or reference cited in this disclosure is hereby incorporated in its entirety by reference. None of the foregoing examples are intended to limit the full scope of the invention as fully set forth in the claims that follow.

1-90. (canceled)

91. A method for screening for a bioactive natural compound produced by a target microorganism comprising:
(a) enriching an environmental sample for the target microorganism by treating the sample with a counter-selective agent that selects against microorganisms not of interest for screening, but present in the sample, thereby obtaining a compound produced by the target microorganism;
(b) culturing a cell in the presence of the compound obtained in step (a), wherein said cell comprises:
(i) a plurality of drug resistance genes, at least two of which are different and which are introduced by artificially recombining them into the chromosome of the cell; and
(ii) one or more genes conferring resistance to said counter-selective agent; and
(c) determining whether the cell grows in the presence of said compound.

92. The method of claim 91, wherein the at least two drug resistance genes which are different are encoded by exogenous, heterologous DNA sequences.

93. The method of claim 91, wherein the first of the at least two different drug resistance genes is introduced by artificially recombining it into an essential chromosomal locus.

94. The method of claim 91, wherein the first of the at least two different drug resistance genes is introduced by artificially recombining it into a non-essential chromosomal locus.

95. The method of claim 91, wherein the second of the at least two different drug resistance genes is introduced by artificially recombining it into a non-essential chromosal locus.

96. The method of claim 91, wherein the cell is a bacterium, a fungal cell, a mammalian cell, a plant cell, or an insect cell.

97. The method of claim 93, wherein the essential chromosomal locus of the cell before recombination encodes at least one gene product involved in the biosynthesis of an essential nutrient and the artificial recombination of the first drug resistance gene into the essential chromosomal locus thereby renders the cell auxotrophic.

98. The method of claim 91, wherein the cell comprises at least one auxotrophoic mutation.

99. The method of claim 91, wherein at least one of the drug resistance genes is a pbp5, blaZ, aph(2″)-Ib, aac(6′)-Im, aac(3′)-IV, TEM-1 bla, cat, nalA37, neo, aadA1, rpsL150, sat, dhfrI, dfrB2, tetA, ble, or vanA drug resistance gene.

100. The method of claim 96, wherein the cell is a Gram-positive or a Gram-negative bacterium.

101. The method of claim 100, wherein the bacterium is a strain of Escherichia coli, Salmonella, Klebsiella, Acinetobacter, Bacillus subtilis, Staphylococcus aureus, Streptococcus pneumoniae, Enterococcus faecalis, Enterococcus faecium, Streptomyces, Amycolatopsis, Saccharopolyspora, Micromonospora, or Streptosporangium.

102. The method of claim 100, wherein at least one of the drug resistance genes confers resistance to ampicillin, an aminoglycoside, apramycin, bleomycin, a β-lactam, chloramphenicol, nalidixic acid, neomycin, spectinomycin, streptomycin, streptothricin, tetracycline, trimethoprim, or vancomycin.

103. The method of claim 96, wherein the cell is a fungal cell selected from: a yeast cell, Saccharomyces cerevisiae, Candida albicans, Aspergillus nidulans, Aspergillus niger, Aspergillus fumigatus, Penicillium, Cephalosporium, or other fungi imperfecta

104. The method of claim 103, wherein at least one of the drug resistance genes confers resistance to itraconazole, azoles, amphotericin, or fluconazole.

105. The method of claim 91, wherein the target microorganism is an actinomycete.

106. The method of claim 91, wherein the environmental sample is a soil sample, an air sample, a deposit near a hot spring, a deposit near a thermal vent, a freshwater filtrate, a marine sediment, an estuarine sediment, or a seawater filtrate.

107. The method of claim 91, wherein said counter-selective agent is nalidixic acid, trimethoprim, cycloheximide, or nystatin.

108. The method of claim 91 wherein in step (a) obtaining said compound comprises encapsulating an individual microorganism that produces the compound into an alginate bead.

109. The method of claim 91, further comprising the step of isolating the target microorganism after determining whether the cell grows in the presence of the compound.


SEQUENCE LISTING (30 sequences)

SEQ ID NO: 1 - DNA, 72 bp, Artificial Sequence
  Description of Artificial Sequence Synthetic primer
atgacaacggacgatcttgcctttgaccaacgccatatctggcacccatacgagatgcttgtttaccggtag

SEQ ID NO: 2 - DNA, 70 bp, Artificial Sequence
  Description of Artificial Sequence Synthetic primer
atgtttcatcctgtaccgcgcggttaaccgctgcggtcagacgctgcaacccagctctctaacgcttgag

SEQ ID NO: 3 - DNA, 70 bp, Artificial Sequence
  Description of Artificial Sequence Synthetic primer
gaaacttttaaaagtagcagcaattgcagcaatcgtattctccggtagcgcaggtcgctcacggtaactg

SEQ ID NO: 4 - DNA, 70 bp, Artificial Sequence
  Description of Artificial Sequence Synthetic primer
cagtggaacggcaaaaattctgaaatgacggttaaacagttcggtggtggtctccgctcatgagctcagc

SEQ ID NO: 5 - DNA, 70 bp, Artificial Sequence
  Description of Artificial Sequence Synthetic primer
ccgaagttgagcaagcgcgccagatgctggcaaaagcgcaaaaaccgatgggcgcgcctacctgtgacgg

SEQ ID NO: 6 - DNA, 70 bp, Artificial Sequence
  Description of Artificial Sequence Synthetic primer
cgtaacgccaggaatgttcatcacgcagctgcgcgcagtgttgctgccagtcatcgcagtactgttgtat

SEQ ID NO: 7 - DNA, 72 bp, Artificial Sequence
  Description of Artificial Sequence Synthetic primer
atagttaccaaatctggcaaaagggttaacaagtggcagcaacggattcgctggcaaatattctgaaatgag

SEQ ID NO: 8 - DNA, 76 bp, Artificial Sequence
  Description of Artificial Sequence Synthetic primer
ggttctcagcatccaatgtttccgccacctgctcagggatcaccgaaatcttcatctatacttcctcctgggtacc

SEQ ID NO: 9 - DNA, 29 bp, Artificial Sequence
  Description of Artificial Sequence Synthetic primer
cggcgcatatggttaacttggacgctgag

SEQ ID NO: 10 - DNA, 32 bp, Artificial Sequence
  Description of Artificial Sequence Synthetic primer
cggcgggatccttacactctccattccatcag

SEQ ID NO: 11 - DNA, 32 bp, Artificial Sequence
  Description of Artificial Sequence Synthetic primer
cggcgggatccgctcatgagacaataaccctg

SEQ ID NO: 12 - DNA, 31 bp, Artificial Sequence
  Description of Artificial Sequence Synthetic primer
cggcggctagcttaccaatgcttaatcagtg

SEQ ID NO: 13 - DNA, 71 bp, Artificial Sequence
  Description of Artificial Sequence Synthetic primer
ggtggccggaaggcgaagcggcatgcatttacgttgacaccatcgaatggaaatcataaaaaatttatttg

SEQ ID NO: 14 - DNA, 71 bp, Artificial Sequence
  Description of Artificial Sequence Synthetic primer
gtacataatggatttccttacgcgaaatacgggcagacatggcctgcccggttaccaatgcttaatcagtg

SEQ ID NO: 15 - DNA, 71 bp, Artificial Sequence
  Description of Artificial Sequence Synthetic primer
gagcctcgcaaattttgtcgttggtgacgggaaaacataaattaatcttgaaatcataaaaaatttatttg

SEQ ID NO: 16 - DNA, 71 bp, Artificial Sequence
  Description of Artificial Sequence Synthetic primer
gttggattattgattgcattactgcctgagattggtcgtctgcaacgccagttaccaatgcttaatcagtg

SEQ ID NO: 17 - DNA, 71 bp, Artificial Sequence
  Description of Artificial Sequence Synthetic primer
cagtcacgacgttgtaaaacgacggccagtgaatccgtaatcatggtcataatcaatccagcccctgtgag

SEQ ID NO: 18 - DNA, 70 bp, Artificial Sequence
  Description of Artificial Sequence Synthetic primer
gcggtatggcatgatagcgcccggaagagagtcaattcagggtggtgaatttaccaatgcttaatcagtg

SEQ ID NO: 19 - DNA, 71 bp, Artificial Sequence
  Description of Artificial Sequence Synthetic primer
ttagaataatttttttgaccagccgagcttggtgcttaatgtgttgaaatttcttagacgtcaggtggcac

SEQ ID NO: 20 - DNA, 71 bp, Artificial Sequence
  Description of Artificial Sequence Synthetic primer
attgctgaccaccagccagaatgcattggcattaatggccgacggtgaagccgatacgcgagcgaacgtga

SEQ ID NO: 21 - DNA, 73 bp, Artificial Sequence
  Description of Artificial Sequence Synthetic primer
cagtcacgacgttgtaaaacgacggccagtgaatccgtaatcatggtcatagtcgttttcctgtatgaaaaac

SEQ ID NO: 22 - DNA, 72 bp, Artificial Sequence
  Description of Artificial Sequence Synthetic primer
cgcggtatggcatgataccgcccggaagagagtcaattcagggtggtgaattaccaatgcttaatcagtgag

SEQ ID NO: 23 - DNA, 72 bp, Artificial Sequence
  Description of Artificial Sequence Synthetic primer
aaacagatcgaagaaggggttgaatcgcaggctattctggtggccggaaggttacactctccattccatcag

SEQ ID NO: 24 - DNA, 72 bp, Artificial Sequence
  Description of Artificial Sequence Synthetic primer
atgagacaataaccctgataaatgcttcaataatattgaaaaaggaagagtaaatcataaaaaatttatttg

SEQ ID NO: 25 - DNA, 71 bp, Artificial Sequence
  Description of Artificial Sequence Synthetic primer
gagcggataacaatttcacacagaattcattaaagaggagaaattaaccatatgagtattcaacatttccg

SEQ ID NO: 26 - DNA, 71 bp, Artificial Sequence
  Description of Artificial Sequence Synthetic primer
aaacagatcgaagaaggggttgaatcgcaggctaaactggtggccggaaggcgaagcggcatgcatttacg

SEQ ID NO: 27 - DNA, 71 bp, Artificial Sequence
  Description of Artificial Sequence Synthetic primer
cgggatcatggtgtcgtagcccagcacggtgaagttattgatacacagcgaaatcataaaaaatttatttg

SEQ ID NO: 28 - DNA, 70 bp, Artificial Sequence
  Description of Artificial Sequence Synthetic primer
acaccgcggggaaacgcttcggcggtaatccatttactgccatggtgattttaccaatgcttaatcagtg

SEQ ID NO: 29 - DNA, 71 bp, Artificial Sequence
  Description of Artificial Sequence Synthetic primer
cgcgctaaatactgcttcaccacaaggaatgcaaatgaagaaattgctccaaatcataaaaaatttatttg

SEQ ID NO: 30 - DNA, 71 bp, Artificial Sequence
  Description of Artificial Sequence Synthetic primer
cgaagccccgtcgtcgtcatcagttacggaaagggttatgaccgttactgttacactctccattccatcag